(19) United States
(12) Patent Application Publication
(10) Pub. No.: US 2019/0157350 A1
(43) Pub. Date: May 23, 2019

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventor: Tomoaki YOSHIOKA, Kanagawa (JP)

(73) Assignee: FUJIFILM Corporation, Tokyo (JP)

(21) Appl. No.: 16/234,616

(22) Filed: Dec. 28, 2018

Related U.S. Application Data
(63) Continuation of application No. PCT/JP2017/021722, filed on Jun. 13, 2017.

(30) Foreign Application Priority Data
Jul. 27, 2016 (JP) 2016-147485

(51) Int. Cl. H01L 27/30; C07D 471/04; H01L 51/00; H01L 51/42; H01L 51/44

(52) U.S. Cl. CPC H01L 27/307; C07D 471/04; H01L 51/442; H01L 51/4273; H01L 51/0072

(57) ABSTRACT

A photoelectric conversion element has a conductive film, a photoelectric conversion film, and a transparent conductive film in this order, in which the photoelectric conversion film contains a compound represented by Formula (1).

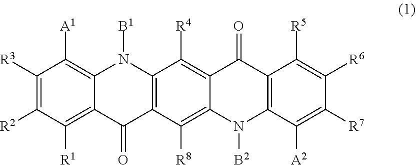

### CROSS-REFERENCE TO RELATED APPLICATIONS

This application is a Continuation of PCT International Application No. PCT/JP2017/021722, filed on Jun. 13, 2017, which claims priority under 35 U.S.C. § 119(a) to Japanese Patent Application No. 2016-147485, filed on Jul. 27, 2016. Each of the above application(s) is hereby expressly incorporated by reference, in its entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In the related art, a planar solid-state imaging element in which photodiodes (PD's) are two-dimensionally arranged and a signal charge generated in each PD is read out by a circuit is widely used as a solid-state imaging element.

In order to realize a color solid-state imaging element, a structure in which color filters transmitting light of a specific wavelength are arranged on a light incident surface side of the planar solid-state imaging element is generally used. Currently, a single plate type solid-state imaging element in which color filters transmitting blue (B) light, green (G) light, and red (R) light are regularly arranged on each of the PD's that have been two-dimensionally arranged is well-known. However, in the single plate type solid-state imaging element, light which has not been transmitted through the color filters is not used, and therefore, light utilization efficiency is poor.

In order to solve these disadvantages, in recent years, development of a photoelectric conversion element having a structure in which an organic photoelectric conversion film is disposed on a substrate for reading out a signal has progressed. US2014/0097416A discloses, for example, a photoelectric conversion element having a photoelectric conversion film containing the following compounds as such a photoelectric conversion element using an organic photoelectric conversion film.

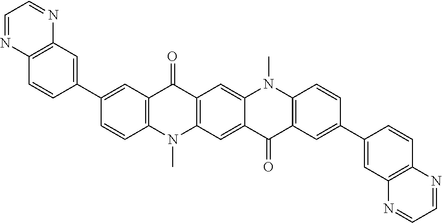 [SMILES: Cn1c2ccc(-c3ccc4nccnc4c3)cc2c(=O)c2cc3c(cc21)c(=O)c1cc(-c2ccc4nccnc4c2)ccc1n3C]

### SUMMARY OF THE INVENTION

In recent years, further improvements are also required for various characteristics required for a photoelectric conversion element to be used in an imaging element and an optical sensor, along with demands for improving performance of the imaging element, the optical sensor, and the like.

For example, further improvement in responsiveness is required.

The present inventor has produced a photoelectric conversion element using a compound (for example, the above-described compound), which is specifically disclosed in US2014/0097416A, and examined responsiveness of the obtained photoelectric conversion element, and as a result, has found that the characteristics thereof do not necessarily reach a level required at the present time and further improvement is required.

In view of the above-described circumstances, an object of the present invention is to provide a photoelectric conversion element exhibiting excellent responsiveness.

Another object of the present invention is to provide an imaging element and an optical sensor which include a photoelectric conversion element. Still another object of the present invention is to provide a compound applied to the above-described photoelectric conversion element.

The present inventor has conducted extensive studies on the above-described problems. As a result, he or she has found that it is possible to solve the above-described problems using a photoelectric conversion film containing a compound (quinacridone) having a predetermined structure, and have completed the present invention.

That is, the above-described problems can be solved by means shown below.

(1) A photoelectric conversion element having a conductive film, a photoelectric conversion film, and a transparent conductive film in this order, in which the photoelectric conversion film contains a compound represented by Formula (1) to be described below.

(2) The photoelectric conversion element according to (1), in which, in Formula (1), B¹ and B² each independently represent any of an alkyl group, an aryl group, or a heteroaryl group.

(3) The photoelectric conversion element according to (1) or (2), in which, in Formula (1), both A¹ and A² represent an aryl group or a heteroaryl group.
(4) The photoelectric conversion element according to any one of (1) to (3), in which a molecular weight of the compound represented by Formula (1) is 470 to 900.
(5) The photoelectric conversion element according to any one of (1) to (4), in which the photoelectric conversion film further includes an n-type organic semiconductor.
(6) The photoelectric conversion element according to any one of (1) to (4), in which the photoelectric conversion film further includes a p-type organic semiconductor.
(7) The photoelectric conversion element according to any one of (1) to (6), further comprising: an electron blocking film.
(8) The photoelectric conversion element according to any one of (1) to (7), further comprising: a hole blocking film.
(9) An optical sensor comprising: the photoelectric conversion element according to any one of (1) to (8).
(10) An imaging element comprising: the photoelectric conversion element according to any one of (1) to (8).
(11) A compound represented by Formula (2) to be described below.

According to the present invention, it is possible to provide a photoelectric conversion element exhibiting excellent responsiveness.

In addition, according to the present invention, it is also possible to provide an imaging element and an optical sensor which include a photoelectric conversion element. Furthermore, according to the present invention, it is also possible to provide a compound to be applied to the above-described photoelectric conversion element.

### BRIEF DESCRIPTION OF THE DRAWINGS

FIG. 1A is a schematic cross-sectional view showing a configuration example of a photoelectric conversion element.

FIG. 1B is a schematic cross-sectional view showing a configuration example of a photoelectric conversion element.

FIG. 2 is a schematic cross-sectional view of one pixel of a hybrid type photoelectric conversion element.

FIG. 3 is a schematic cross-sectional view of one pixel of an imaging element.

FIG. 4 is a ¹H Nuclear Magnetic Resonance (NMR) chart of a compound (D-10).

FIG. 5 is a ¹H Nuclear Magnetic Resonance (NMR) chart of a compound (D-11).

FIG. 6 is a ¹H Nuclear Magnetic Resonance (NMR) chart of a compound (D-12).

FIG. 7 is a ¹H Nuclear Magnetic Resonance (NMR) chart of a compound (D-13).

FIG. 8 is a ¹H Nuclear Magnetic Resonance (NMR) chart of a compound (D-14).

### DESCRIPTION OF THE PREFERRED EMBODIMENTS

Hereinafter, suitable embodiments of a photoelectric conversion element of the present invention will be described.

In the present specification, a substituent (preferably a substituent W to be described below) may be further substituted with a substituent or the like within the scope not impairing an intended effect for which whether it is substituted or unsubstituted is not specified. For example, the expression “alkyl group” corresponds to an alkyl group with which a substituent (preferably a substituent W) may be substituted.

In addition, in the present specification, the numerical range represented by “to” means a range including numerical values denoted before and after “to” as a lower limit value and an upper limit value.

An example of a characteristic point of the present invention compared with the technique in the related art includes a point that a compound having a predetermined structure (hereinafter, also simply referred to as a “specific quinacridone compound”) is used. A specific functional group is introduced into this specific quinacridone compound at a specific position, and as a result, the characteristics (responsiveness) of a photoelectric conversion element having a photoelectric conversion film containing this specific quinacridone compound are improved.

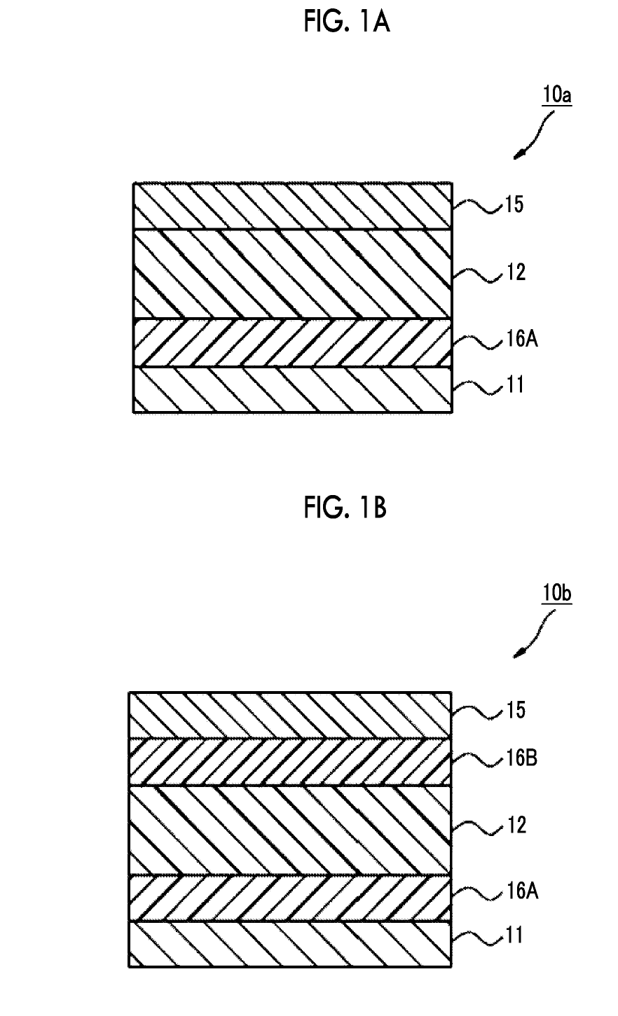

Hereinafter, suitable embodiments of the photoelectric conversion element of the present invention will be described with reference to the drawings. A schematic cross-sectional view of an embodiment of a photoelectric conversion element of the present invention is shown in FIG. 1.

A photoelectric conversion element 10a shown in FIG. 1A has a configuration in which a conductive film (hereinafter, also referred to as a lower electrode) 11 functioning as the lower electrode, an electron blocking film 16A, a photoelectric conversion film 12 containing a compound represented by Formula (1) to be described below, and a transparent conductive film (hereinafter, also referred to as an upper electrode) 15 functioning as the upper electrode are laminated in this order.

A configuration example of another photoelectric conversion element is shown in FIG. 1B. A photoelectric conversion element 10b shown in FIG. 1B has a configuration in which an electron blocking film 16A, a photoelectric conversion film 12, a hole blocking film 16B, and an upper electrode 15 are laminated on a lower electrode 11 in this order. The lamination order of the electron blocking film 16A, the photoelectric conversion film 12, and the hole blocking film 16B in FIGS. 1A and 1B may be appropriately changed in accordance with the application and characteristics.

In the configuration of the photoelectric conversion element 10a (or 10b), light is preferably incident on the photoelectric conversion film 12 through the upper electrode 15.

In addition, in a case of using the photoelectric conversion element 10a (or 10b), a voltage can be applied. In this case, it is preferable that the lower electrode 11 and the upper electrode 15 form a pair of electrodes and a voltage of 1×10⁻⁵ to 1×10⁷ V/cm is applied between this pair of electrodes. From the viewpoints of performance and power consumption, a voltage of 1×10⁻⁴ to 1×10⁷ V/cm is more preferable and a voltage of 1×10⁻³ to 5×10⁶ V/cm is still more preferable.

As the voltage application method, application of a voltage is preferably performed such that the electron blocking film 16A side in FIGS. 1A and 1B becomes a cathode and the photoelectric conversion film 12 side becomes an anode. It is possible to apply a voltage through the same method in cases where the photoelectric conversion element 10a (or 10b) is used as an optical sensor or is incorporated into an imaging element.

As will be described in detail below, the photoelectric conversion element 10a (or 10b) can be suitably applied to applications of the imaging element and the optical sensor.

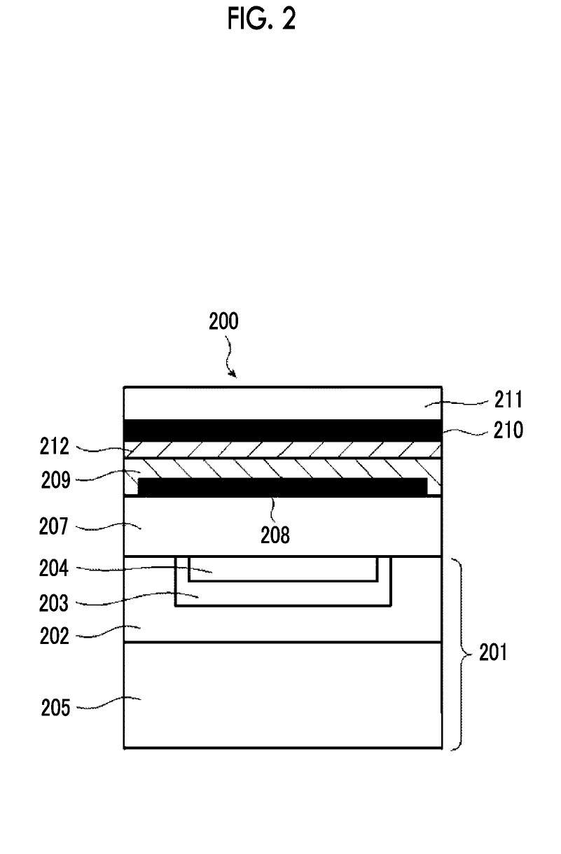

In addition, a schematic cross-sectional view of another embodiment of a photoelectric conversion element of the present invention is shown in FIG. 2.

A photoelectric conversion element 200 shown in FIG. 2 is a hybrid type photoelectric conversion element including an organic photoelectric conversion film 209 and an inorganic photoelectric conversion film 201. The organic photoelectric conversion film 209 includes a compound represented by Formula (1) to be described below.

The inorganic photoelectric conversion film 201 has an n-type well 202, a p-type well 203, and an n-type well 204 on a p-type silicon substrate 205.

Blue light is photoelectrically converted (B pixel) at a p-n junction formed between the p-type well 203 and the n-type well 204 and red light is photoelectrically converted (R pixel) at a p-n junction formed between the p-type well 203 and the n-type well 202. The conduction types of the n-type well 202, the p-type well 203, and the n-type well 204 are not limited thereto.

Furthermore, a transparent insulating layer 207 is disposed on the inorganic photoelectric conversion film 201.

A transparent pixel electrode 208 divided for each pixel is disposed on the insulating layer 207. An organic photoelectric conversion film 209 which absorbs green light and performs photoelectric conversion is disposed on the transparent pixel electrode in a single layer configuration commonly for each pixel. An electron blocking film 212 is disposed on the organic photoelectric conversion film in a single layer configuration commonly for each pixel. A transparent common electrode 210 with a single layer configuration is disposed on the electron blocking film. A transparent protective film 211 is disposed on the uppermost layer. The lamination order of the electron blocking film 212 and the organic photoelectric conversion film 209 may be reversed from that in FIG. 2, and the common electrode 210 may be disposed so as to be divided for each pixel.

The organic photoelectric conversion film 209 constitutes a G pixel for detecting green light.

The pixel electrode 208 is the same as the lower electrode 11 of the photoelectric conversion element 10a shown in FIG. 1A. The common electrode 210 is the same as the upper electrode 15 of the photoelectric conversion element 10a shown in FIG. 1A.

In a case where light from a subject is incident on the photoelectric conversion element 200, green light in the incident light is absorbed by the organic photoelectric conversion film 209 to generate optical charges. The optical charges flow into and accumulate in a green signal charge accumulation region not shown in the drawing from the pixel electrode 208.

The mixed light of the blue light and the red light transmitted through the organic photoelectric conversion film 209 enters the inorganic photoelectric conversion film 201. The blue light having a short wavelength is photoelectrically converted mainly at a shallow portion (in the vicinity of a p-n junction formed between the p-type well 203 and the n-type well 204) of a semiconductor substrate (inorganic photoelectric conversion film) 201 to generate optical charges, and a signal is output to the outside. The red light having a long wavelength is photoelectrically converted mainly at a deep portion (in the vicinity of a p-n junction formed between the p-type well 203 and the n-type well 202) of the semiconductor substrate (inorganic photoelectric conversion film) 201 to generate optical charges, and a signal is output to the outside.

In a case where the photoelectric conversion element 200 is used in an imaging element, a signal readout circuit (an electric charge transfer path in a case of a charge coupled device (CCD) type or a metal-oxide-semiconductor (MOS) transistor circuit in a case of a complementary metal oxide semiconductor (CMOS) type) or a green signal charge accumulation region is formed in a surface portion of the p-type silicon substrate 205. In addition, the pixel electrode 208 is connected to the corresponding green signal charge accumulation region through vertical wiring.

Hereinafter, the form of each layer constituting the photoelectric conversion element of the present invention will be described in detail.

[Photoelectric Conversion Film]

(Compound Represented by Formula (1))

The photoelectric conversion film 12 (or the organic photoelectric conversion film 209) is a film containing a compound represented by Formula (1) as a photoelectric conversion material. A photoelectric conversion element exhibiting excellent responsiveness can be obtained using this compound.

Hereinafter, the compound represented by Formula (1) will be described in detail.

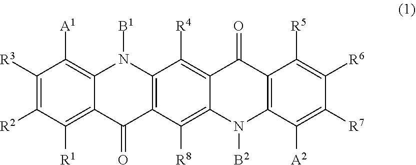

In Formula (1), R¹ to R⁸ each independently represent a hydrogen atom or a substituent. The definition of the above-described substituent is synonymous with the substituent W to be described below. In particular, R¹ to R⁸ each independently represent preferably a hydrogen atom, an alkyl group, an alkoxy group, or a halogen atom and more preferably a hydrogen atom from the viewpoint of obtaining superior responsiveness of the photoelectric conversion element (hereinafter, also simply referred to as the viewpoint of obtaining a superior effect of the present invention”).

Adjacent groups among R¹ to R⁸, A¹, A², B¹, and B² may be linked to each other to form a ring. The type of ring to be formed is not particularly limited. It may be an aromatic ring or a non-aromatic ring, and it is preferably an aromatic ring. In addition, the ring may be a monocyclic ring or a condensed ring consisting of two or more rings. In addition, the aromatic ring may be an aromatic hydrocarbon ring or an aromatic heterocyclic ring.

B¹ and B² each independently represent a hydrogen atom or a substituent. The definition of the above-described substituent is synonymous with the substituent W to be described below.

In particular, from the viewpoint of obtaining a superior effect of the present invention, it is preferable that B¹ and B² each independently represent an alkyl group, an aryl group, or a heteroaryl group, it is more preferable that both B¹ and B² represent an alkyl group, an aryl group, or a heteroaryl group, and it is still more preferable that both B¹ and B² represent an alkyl group.

In addition, from the viewpoint of obtaining a superior effect of the present invention, it is preferable that B¹ and B² are the same groups as each other. For example, a case is exemplified in which both B¹ and B² represent a methyl group.

The number of carbon atoms in an alkyl group is not particularly limited, but is preferably 1 to 10, more preferably 1 to 6, still more preferably 1 to 3, and particularly preferably 1 from the viewpoint of obtaining a superior effect of the present invention. The alkyl group may be linear, branched, or cyclic.

Examples of the alkyl group include a methyl group, an ethyl group, an n-propyl group, an i-propyl group, an n-butyl group, an n-hexyl group, and a cyclohexyl group.

The number of carbon atoms in an aryl group is not particularly limited, but is preferably 6 to 30 and more preferably 6 to 18 from the viewpoint of obtaining a superior effect of the present invention. The aryl group may have a monocyclic structure or a condensed ring structure in which two or more rings are condensed. In addition, a substituent (preferably a substituent W to be described below) may be substituted with the aryl group.

Examples of the aryl group include a phenyl group, a naphthyl group, an anthryl group, a pyrenyl group, a phenanthrenyl group, a methylphenyl group, a dimethylphenyl group, a biphenyl group, and a fluorenyl group, and a phenyl group, a naphthyl group, or an anthryl group is preferable.

The number of carbon atoms in a heteroaryl group (monovalent aromatic heterocyclic group) is not particularly limited, but is preferably 3 to 30 and more preferably 3 to 18 from the viewpoint of obtaining a superior effect of the present invention. In addition, a substituent (preferably a substituent W to be described below) may be substituted with the heteroaryl group.

A hetero atom is included in the heteroaryl group in addition to a carbon atom and a hydrogen atom. Examples of the hetero atom include a nitrogen atom, a sulfur atom, an oxygen atom, a selenium atom, a tellurium atom, a phosphorus atom, a silicon atom, and a boron atom. A nitrogen atom, a sulfur atom, or an oxygen atom is preferable.

The number of hetero atoms contained in a heteroaryl group is not particularly limited, but is usually about 1 to 10 preferably 1 to 4, and more preferably 1 or 2.

The number of ring members of a heteroaryl group is not particularly limited, but is preferably a 3- to 8-membered ring, more preferably a 5- to 7-membered ring, and still more preferably a 5- to 6-membered ring. The heteroaryl group may have a monocyclic structure or a condensed ring structure in which two or more rings are condensed. In the case of the condensed ring structure, an aromatic hydrocarbon ring (for example, a benzene ring) which does not contain a hetero atom may be contained therein.

Examples of the heteroaryl group include a pyridyl group, a quinolyl group, an isoquinolyl group, an acridinyl group, a phenanthridinyl group, a pteridinyl group, a pyrazinyl group, a quinoxalinyl group, a pyrimidinyl group, a quinazolyl group, a pyridazinyl group, a cinnolinyl group, a phthalazinyl group, a triazinyl group, an oxazolyl group, a benzoxazolyl group, a thiazolyl group, a benzothiazolyl group, an imidazolyl group, a benzimidazolyl group, a pyrazolyl group, an indazolyl group, an isoxazolyl group, a benzisoxazolyl group, an isothiazolyl group, a benzisothiazolyl group, an oxadiazolyl group, a thiadiazolyl group, a triazolyl group, a tetrazolyl group, a furyl group, a benzofuryl group, a thienyl group, a benzothienyl group, a dibenzofuryl group, a dibenzothienyl group, a pyrrolyl group, an indolyl group, an imidazopyridinyl group, and a carbazolyl group.

A¹ and A² each independently represent a hydrogen atom or a substituent, at least one of A¹ or A² represents an aryl group or a heteroaryl group, and

The definitions and suitable aspects of the aryl group or heteroaryl group represented by A¹ and A² are the same as those of the aryl group and heteroaryl group represented by B¹ and B².

In particular, from the viewpoint of obtaining a superior effect of the present invention, it is preferable that both A¹ and A² are aryl groups or heteroaryl groups.

In addition, from the viewpoint of obtaining a superior effect of the present invention, it is preferable that A¹ and A² represent the same group as each other. For example, a case is exemplified in which both A¹ and A² represent a phenyl group.

In particular, an example of a suitable aspect of the compound represented by Formula (1) includes a compound represented by Formula (2) from the viewpoint from the viewpoint of obtaining a superior effect of the present invention.

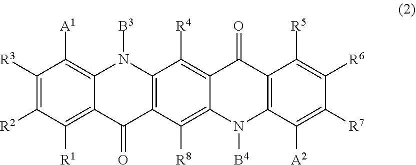

In Formula (2), R¹ to R⁸ each independently represent a hydrogen atom or a substituent. B³ and B⁴ each independently represent any of an alkyl group, an aryl group, or a heteroaryl group, A¹ and A² each independently represent a hydrogen atom or a substituent, at least one of A¹ or A² represents an aryl group or a heteroaryl group, and

The definitions and suitable aspects of R¹ to R⁸, A¹, and A² in Formula (2) are the same as those of R¹ to R⁸, A¹, and A² in Formula (1).

The definitions and suitable aspects of the alkyl group, aryl group, and heteroaryl group represented by B³ and B⁴ are the same as those of each of the groups described in by B¹ and B² above. Among these, from the viewpoint of obtaining a superior effect of the present invention, both B³ and B⁴ are preferably alkyl groups, and more preferably methyl groups.

In addition, from the viewpoint of obtaining a superior effect of the present invention, it is preferable that B³ and B⁴ are the same groups as each other. For example, a case is exemplified in which both B³ and B⁴ represent a methyl group.

In addition, adjacent groups among R¹ to R⁸, A¹, A², B³, and B⁴ may be linked to each other to form a ring. Examples of rings formed by linking adjacent groups among R¹ to R⁸, A¹, A², B³ and B⁴ to each other include the forms described in the rings formed by linking adjacent groups among R¹ to R⁸, A¹, A², B¹ and B² to each other.

The substituent W in the present specification will be described below.

Examples of the substituent W include a halogen atom, an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group, a cyano group, a hydroxy group, a nitro group, a carboxy group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonium group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a heterocyclic thio group, a sulfamoyl group, a sulfo group, an alkyl- or arylsulfinyl group, an alkyl- or arylsulfonyl group, an acyl group, an aryloxycarbonyl group, an alkoxycarbonyl group, A carbamoyl group, an aryl- or heterocyclic azo group, an imide group, a phosphino group, a phosphinyl group, a phosphinyloxy group, a phosphinylamino group, a phosphono group, a silyl group, a hydrazino group, a ureido group, a boronic acid group (—B(OH)₂), a phosphato group (—OPO(OH)₂), a sulfato group (—OSO₃H), and other well-known substituents.

In addition, the substituent W may be further substituted with a substituent W. For example, a halogen atom may be substituted with an alkyl group.

The details of the substituent W are disclosed in paragraph [0023] of JP2007-234651A.

Hereinafter, the compound represented by Formula (1) will be exemplified.

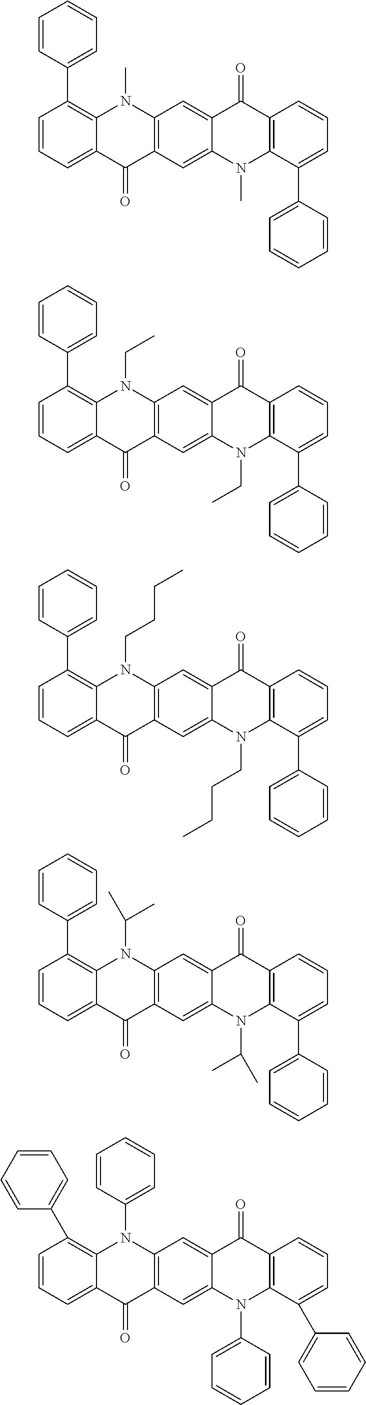 [SMILES: Cn1c2cc3c(=O)c4cccc(-c5ccccc5)c4n(C)c3cc2c(=O)c2cccc(-c3ccccc3)c21 ; CCn1c2cc3c(=O)c4cccc(-c5ccccc5)c4n(CC)c3cc2c(=O)c2cccc(-c3ccccc3)c21 ; CCCCn1c2cc3c(=O)c4cccc(-c5ccccc5)c4n(CCCC)c3cc2c(=O)c2cccc(-c3ccccc3)c21 ; CC(C)n1c2cc3c(=O)c4cccc(-c5ccccc5)c4n(C(C)C)c3cc2c(=O)c2cccc(-c3ccccc3)c21 ; O=c1c2cc3c(cc2n(-c2ccccc2)c2c(-c4ccccc4)cccc12)c(=O)c1cccc(-c2ccccc2)c1n3-c1ccccc1]

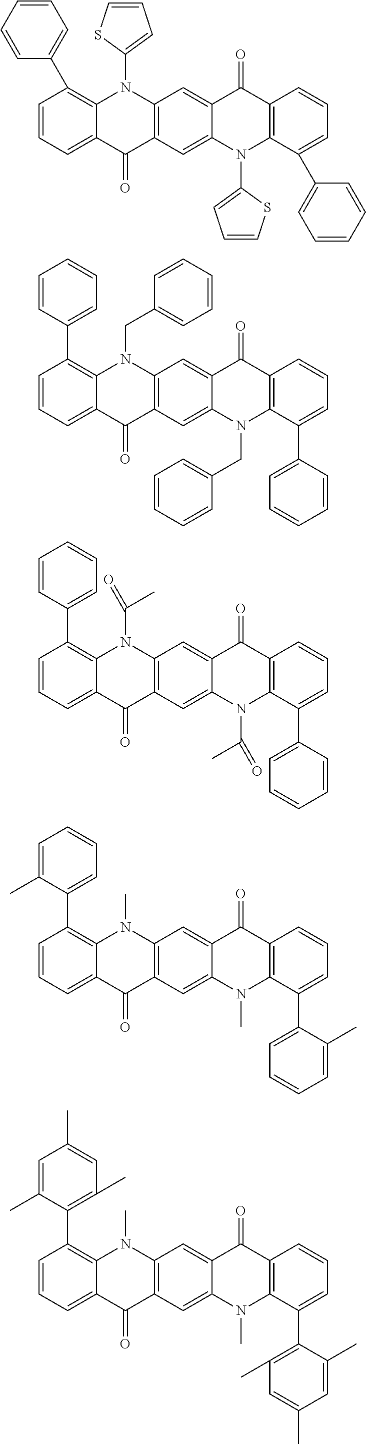

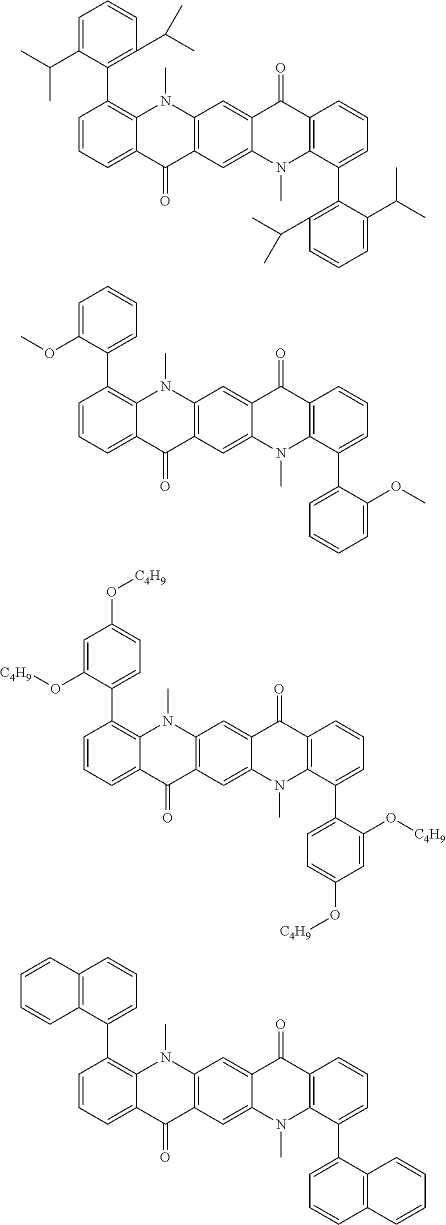

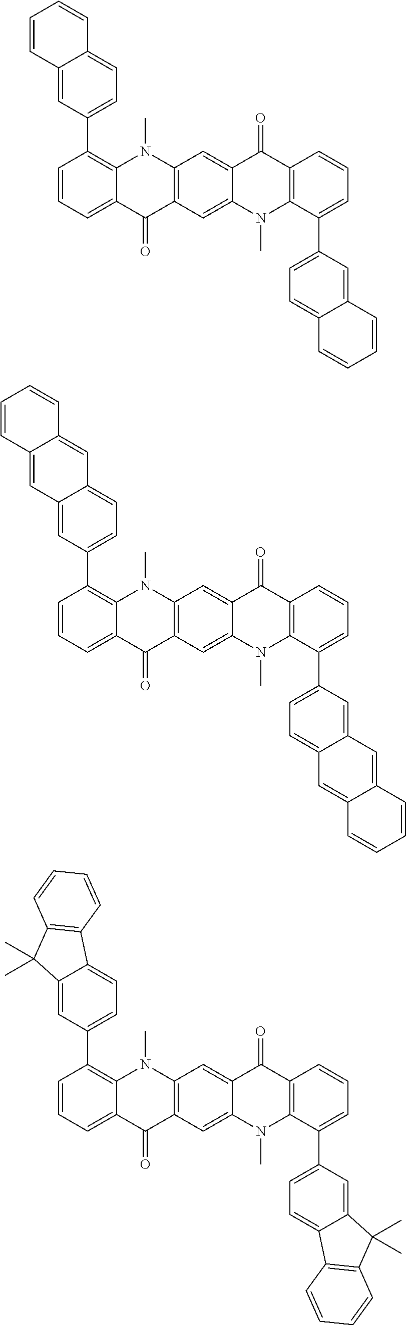

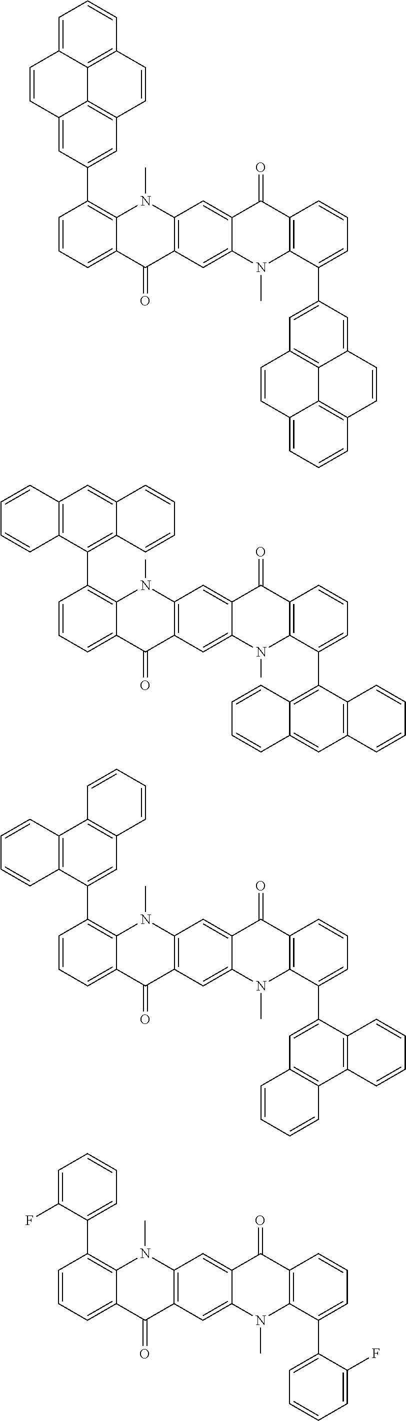 [SMILES: Cn1c2cc3c(=O)c4cccc(-c5cc6ccc7cccc8ccc(c5)c6c78)c4n(C)c3cc2c(=O)c2cccc(-c3cc4ccc5cccc6ccc(c3)c4c56)c21 ; Cn1c2cc3c(=O)c4cccc(-c5c6ccccc6cc6ccccc56)c4n(C)c3cc2c(=O)c2cccc(-c3c4ccccc4cc4ccccc34)c21 ; Cn1c2cc3c(=O)c4cccc(-c5cc6ccccc6c6ccccc56)c4n(C)c3cc2c(=O)c2cccc(-c3cc4ccccc4c4ccccc34)c21 ; Cn1c2cc3c(=O)c4cccc(-c5ccccc5F)c4n(C)c3cc2c(=O)c2cccc(-c3ccccc3F)c21]

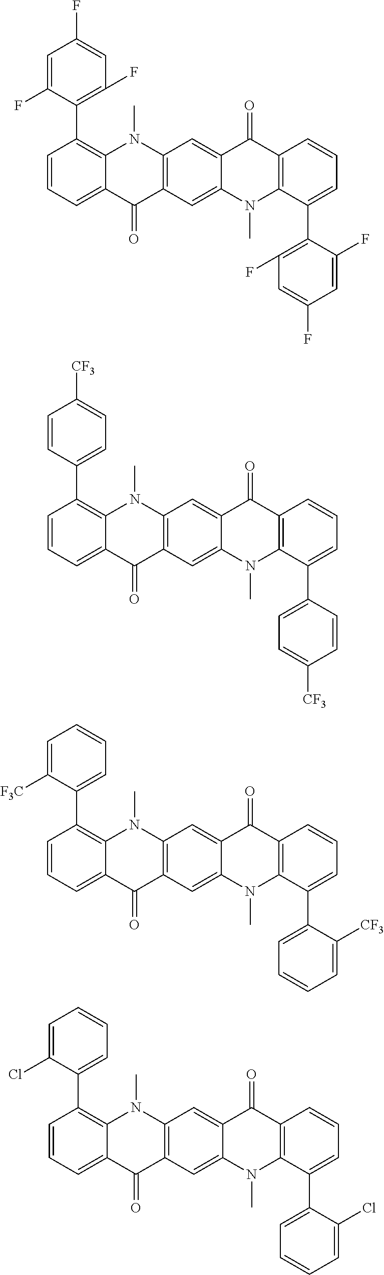 [SMILES: Cn1c2cc3c(=O)c4cccc(-c5c(F)cc(F)cc5F)c4n(C)c3cc2c(=O)c2cccc(-c3c(F)cc(F)cc3F)c21 ; Cn1c2cc3c(=O)c4cccc(-c5ccc(C(F)(F)F)cc5)c4n(C)c3cc2c(=O)c2cccc(-c3ccc(C(F)(F)F)cc3)c21 ; Cn1c2cc3c(=O)c4cccc(-c5ccccc5C(F)(F)F)c4n(C)c3cc2c(=O)c2cccc(-c3ccccc3C(F)(F)F)c21 ; Cn1c2cc3c(=O)c4cccc(-c5ccccc5Cl)c4n(C)c3cc2c(=O)c2cccc(-c3ccccc3Cl)c21]

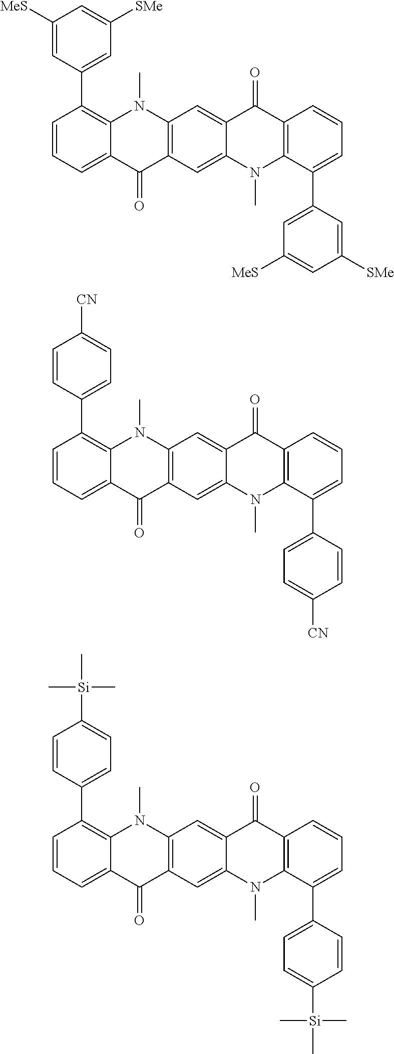

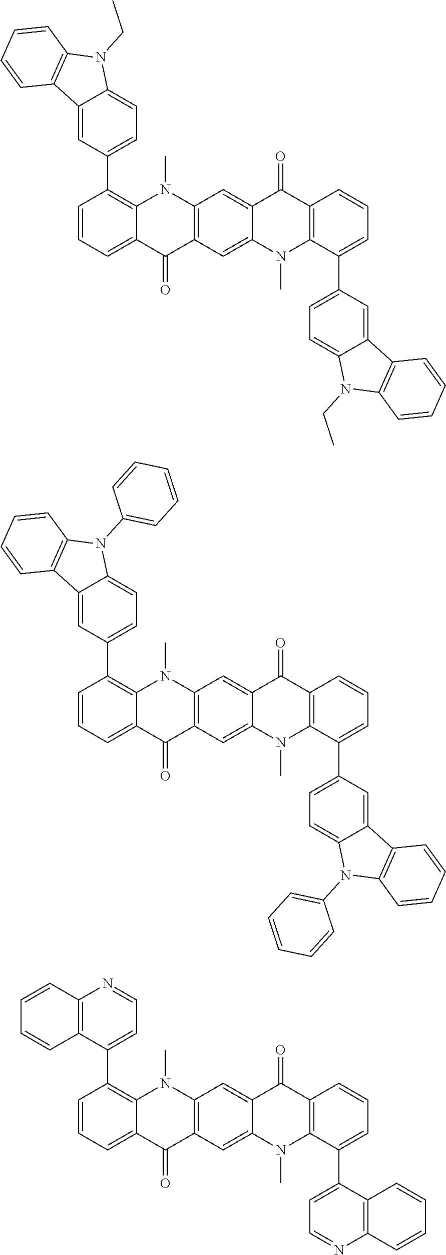 [SMILES: CCn1c2ccccc2c2cc(-c3cccc4c(=O)c5cc6c(cc5n(C)c34)c(=O)c3cccc(-c4ccc5c(c4)c4ccccc4n5CC)c3n6C)ccc21 ; Cn1c2cc3c(=O)c4cccc(-c5ccc6c(c5)c5ccccc5n6-c5ccccc5)c4n(C)c3cc2c(=O)c2cccc(-c3ccc4c(c3)c3ccccc3n4-c3ccccc3)c21 ; Cn1c2cc3c(=O)c4cccc(-c5ccnc6ccccc56)c4n(C)c3cc2c(=O)c2cccc(-c3ccnc4ccccc34)c21]

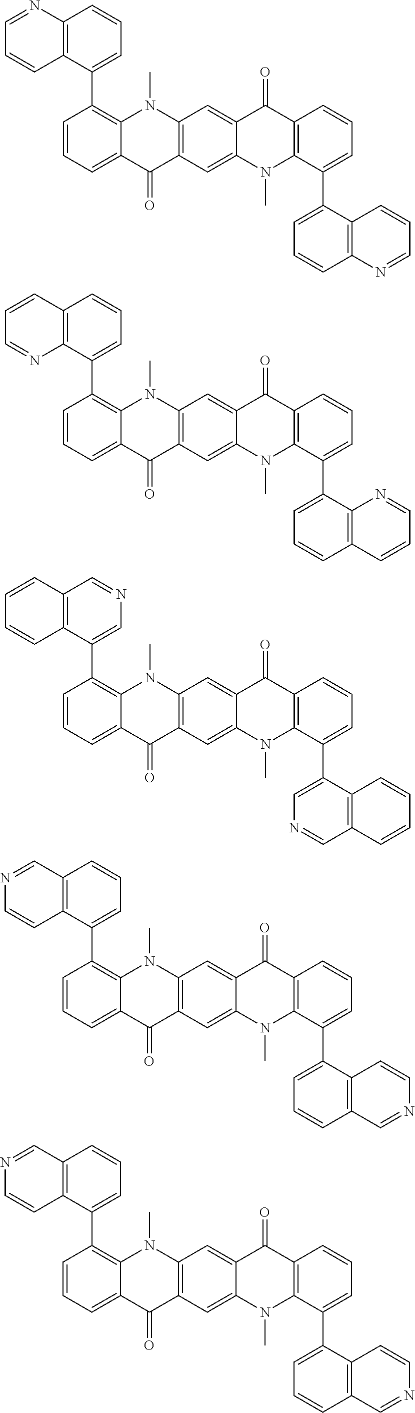 [SMILES: Cn1c2cc3c(=O)c4cccc(-c5cccc6ncccc56)c4n(C)c3cc2c(=O)c2cccc(-c3cccc4ncccc34)c21 ; Cn1c2cc3c(=O)c4cccc(-c5cccc6cccnc56)c4n(C)c3cc2c(=O)c2cccc(-c3cccc4cccnc34)c21 ; Cn1c2cc3c(=O)c4cccc(-c5cncc6ccccc56)c4n(C)c3cc2c(=O)c2cccc(-c3cncc4ccccc34)c21 ; Cn1c2cc3c(=O)c4cccc(-c5cccc6cnccc56)c4n(C)c3cc2c(=O)c2cccc(-c3cccc4cnccc34)c21 ; Cn1c2cc3c(=O)c4cccc(-c5cccc6cnccc56)c4n(C)c3cc2c(=O)c2cccc(-c3cccc4cnccc34)c21]

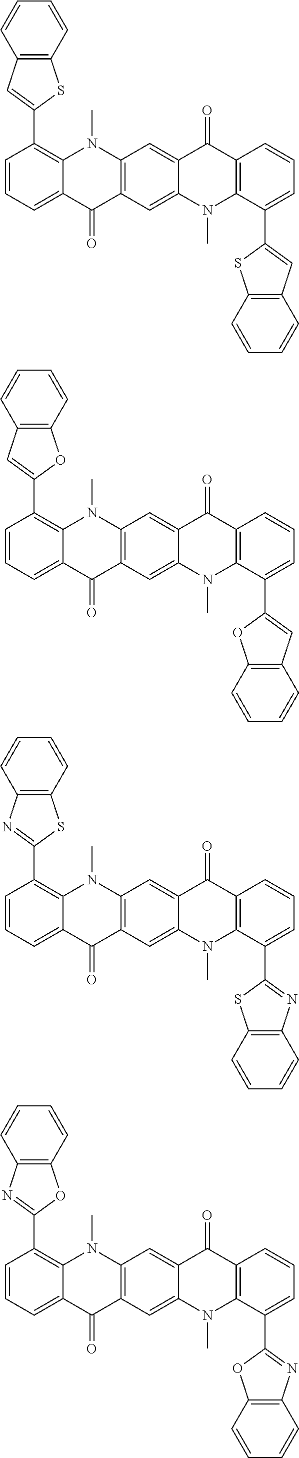 [SMILES: Cn1c2cc3c(=O)c4cccc(-c5cc6ccccc6s5)c4n(C)c3cc2c(=O)c2cccc(-c3cc4ccccc4s3)c21 ; Cn1c2cc3c(=O)c4cccc(-c5cc6ccccc6o5)c4n(C)c3cc2c(=O)c2cccc(-c3cc4ccccc4o3)c21 ; Cn1c2cc3c(=O)c4cccc(-c5nc6ccccc6s5)c4n(C)c3cc2c(=O)c2cccc(-c3nc4ccccc4s3)c21 ; Cn1c2cc3c(=O)c4cccc(-c5nc6ccccc6o5)c4n(C)c3cc2c(=O)c2cccc(-c3nc4ccccc4o3)c21]

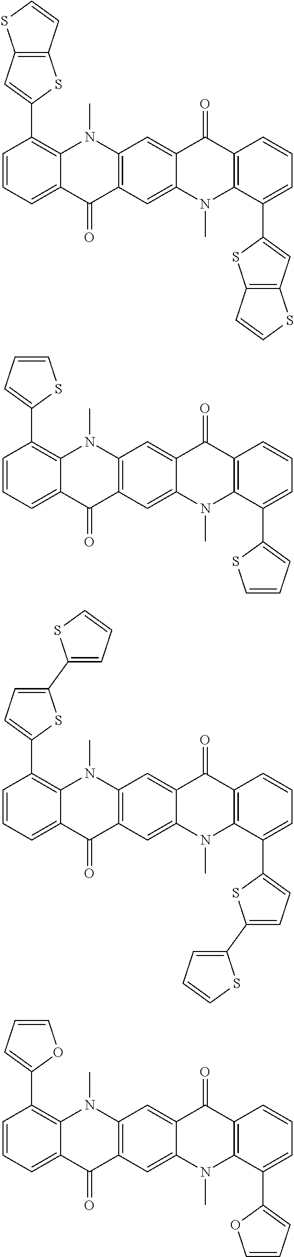 [SMILES: Cn1c2cc3c(=O)c4cccc(-c5cc6sccc6s5)c4n(C)c3cc2c(=O)c2cccc(-c3cc4sccc4s3)c21 ; Cn1c2cc3c(=O)c4cccc(-c5cccs5)c4n(C)c3cc2c(=O)c2cccc(-c3cccs3)c21 ; Cn1c2cc3c(=O)c4cccc(-c5ccc(-c6cccs6)s5)c4n(C)c3cc2c(=O)c2cccc(-c3ccc(-c4cccs4)s3)c21 ; Cn1c2cc3c(=O)c4cccc(-c5ccco5)c4n(C)c3cc2c(=O)c2cccc(-c3ccco3)c21]

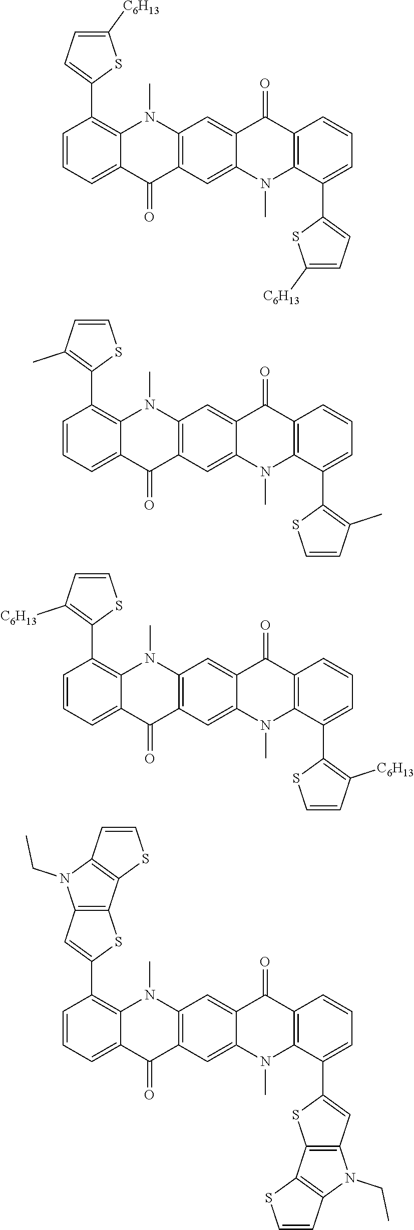

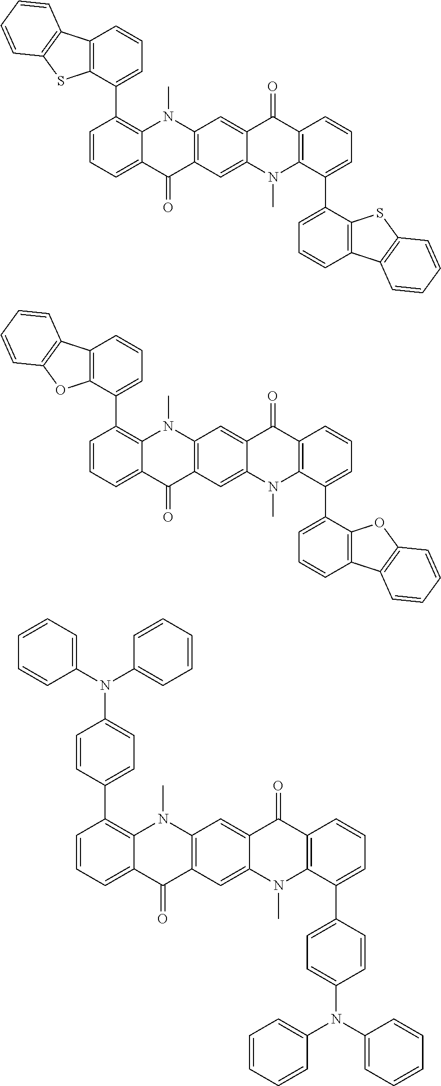 [SMILES: Cn1c2cc3c(=O)c4cccc(-c5cccc6c5sc5ccccc56)c4n(C)c3cc2c(=O)c2cccc(-c3cccc4c3sc3ccccc34)c21 ; Cn1c2cc3c(=O)c4cccc(-c5cccc6c5oc5ccccc56)c4n(C)c3cc2c(=O)c2cccc(-c3cccc4c3oc3ccccc34)c21 ; Cn1c2cc3c(=O)c4cccc(-c5ccc(N(c6ccccc6)c6ccccc6)cc5)c4n(C)c3cc2c(=O)c2cccc(-c3ccc(N(c4ccccc4)c4ccccc4)cc3)c21]

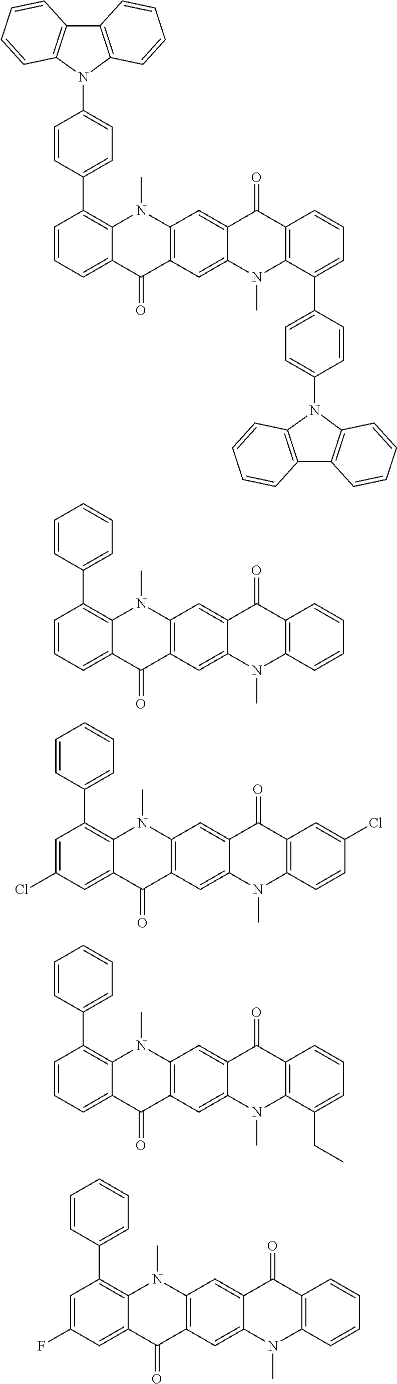

The molecular weight of the compound represented by Formula (1) is not particularly limited, but is preferably 470 to 900. In a case where the molecular weight is less than or equal to 900, the vapor deposition temperature does not increase, and therefore, decomposition of the compound hardly occurs. In a case where the molecular weight is greater than or equal to 470, the glass transition point of a vapor deposition does not decrease, and therefore, the heat resistance of the photoelectric conversion element is improved.

The compound represented by Formula (1) is preferably a compound in which an ionization potential in a single film is −5.0 to −6.0 eV from the viewpoints of stability in a case of using the compound as a p-type organic semiconductor and matching of energy levels between the compound and an n-type organic semiconductor.

The compound represented by Formula (1) is particularly useful as a material for a photoelectric conversion film used for an imaging element, an optical sensor, or a photoelectric cell. In many cases, the compound represented by Formula (1) usually functions as a p-type organic compound (p-type organic semiconductor) within the photoelectric conversion film. In addition, the compound represented by Formula (1) can also be used as a coloring material, a liquid crystal material, an organic semiconductor material, an organic light emitting element material, a charge transport material, a pharmaceutical material, and a fluorescent diagnostic drug material.

(Other Materials)

The photoelectric conversion film may contain components other than the above-described compound represented by Formula (1). For example, the photoelectric conversion film may contain an n-type organic semiconductor or a p-type organic semiconductor.

The n-type organic semiconductor is an acceptor organic semiconductor material (compound) and refers to an organic compound having a property of easily accepting electrons. More specifically, the n-type organic semiconductor refers to an organic compound having a higher electron affinity in a case where two organic compounds are brought into contact with each other.

Examples of the n-type organic semiconductor include a condensed aromatic carbocyclic compound (for example, a naphthalene derivative, an anthracene derivative, a phenanthrene derivative, a tetracene derivative, a pyrene derivative, a perylene derivative, and a fluoranthene derivative), 5- to 7-membered heterocyclic compounds (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole) which contain at least one of a nitrogen atom, an oxygen atom, or a sulfur atom, a polyarylene compound, a fluorene compound, a cyclopentadiene compound, a silyl compound, and a metal complex having a nitrogen-containing heterocyclic compound as a ligand.

The p-type organic semiconductor is a donor organic semiconductor material (compound) and refers to an organic compound having a property of easily donating electrons. More specifically, the p-type organic semiconductor refers to an organic compound having a smaller ionization potential in a case where two organic compounds are brought into contact with each other.

Examples of the p-type organic semiconductor include a triarylamine compound, a benzidine compound, a pyrazoline compound, a styrylamine compound, a hydrazone compound, a carbazole compound, a polysilane compound, a thiophene compound, a cyanine compound, an oxonol compound, a polyamine compound, an indole compound, a pyrrole compound, a pyrazole compound, a polyarylene compound, a condensed aromatic carbocyclic compound, and a metal complex having a nitrogen-containing heterocyclic compound as a ligand.

Any organic coloring agent may be used as an n-type organic semiconductor or a p-type organic semiconductor. Examples thereof include a cyanine coloring agent, a styryl coloring agent, a hemicyanine coloring agent, a merocyanine coloring agent (containing zero methine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a fulgide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, and a metal complex coloring agent.

On the other hand, in the case of the form shown in FIG. 2, it is preferable that the n-type organic semiconductor and the p-type organic semiconductor are colorless or have an absorption maximum wavelength and/or an absorption waveform close to the compound represented by Formula (1), and it is preferable that the specific numerical value of the absorption maximum wavelength is less than or equal to 400 nm or 500 nm to 600 nm.

The photoelectric conversion film preferably has a bulk hetero structure which is formed in a state in which the above-described compound represented by Formula (1) is mixed with the n-type organic semiconductor or the p-type organic semiconductor. The bulk hetero structure is a layer in which an n-type organic semiconductor and a p-type organic semiconductor are mixed with each other and dispersed in a photoelectric conversion film. A photoelectric conversion film having the bulk hetero structure can be formed through either a wet method or a dry method. The bulk hetero structure is described in detail in <0013> to <0014> of JP-2005-303266A.

The content of the compound represented by Formula (1) to the total content of the compound represented by Formula (1) and the n-type organic semiconductor or the p-type organic semiconductor (=film thickness in terms of single layer of compound represented by Formula (1)/(film thickness in terms of single layer of compound represented by Formula (1)+film thickness in terms of single layer of n-type organic semiconductor or p-type organic semiconductor)×100) is preferably 20 to 80 volume %, more preferably 30 to 70 volume %, and still more preferably 40 to 60 volume % from the viewpoint of responsiveness of the photoelectric conversion element.

The photoelectric conversion film containing the compound represented by Formula (1) is non-luminescent film and has characteristics different from those of an organic electric field light emitting element (OLED). The non-luminescent film means a film having a luminescence quantum efficiency of less than or equal to 1%. The luminescence quantum efficiency is preferably less than or equal to 0.5% and more preferably less than or equal to 0.1%.

(Film Forming Method)

The photoelectric conversion film can be formed mainly through a dry film formation method. Specific examples of the dry film formation method include physical vapor phase growth methods such as a vapor deposition method (particularly a vacuum vapor deposition method), a sputtering method, an ion plating method, and a molecular beam epitaxy (MBE) method, or chemical vapor deposition (CVD) methods such as plasma polymerization. Among these, the vacuum vapor deposition method is preferable. In a case where a photoelectric conversion film is formed through the vacuum vapor deposition method, it is possible to set the manufacturing conditions such as the vacuum degree and the vapor deposition temperature in accordance with a usual method.

The thickness of a photoelectric conversion film is preferably 10 to 1,000 nm, more preferably 50 to 800 nm, and still more preferably 100 to 500 nm.

[Electrode]

The electrodes (the upper electrode (transparent conductive film) 15 and the lower electrode (conductive film) 11) are made of a conductive material. Examples of the conductive material include metal, alloy, a metal oxide, an electrically conductive compound, and a mixture thereof.

Since light is incident from the upper electrode 15, it is preferable that the upper electrode 15 is transparent for light to be detected. Examples of the material forming the upper electrode 15 include conductive metal oxides such as tin oxide (ATO, FTO) doped with antimony, fluorine, or the like, tin oxide, zinc oxide, indium oxide, indium tin oxide (ITO), and indium zinc oxide (IZO), metal thin films such as gold, silver, chromium, and nickel, mixtures or laminates of these metals and the conductive metal oxides, and organic conductive materials such as polyaniline, polythiophene, and polypyrrole. Among these, conductive metal oxides are preferable from the viewpoints of high conductivity, transparency, and the like.

In general, in a case where a conductive film is made to be thinner than a certain range, a resistance value is rapidly increased. In the solid-state imaging element into which the photoelectric conversion element according to the present embodiment is incorporated, the sheet resistance is preferably 100 to 10,000Ω/□, and the degree of freedom of the range of the film thickness that can be thinned is large. In addition, as the thickness of the upper electrode (transparent conductive film) 15 is thinner, the amount of light absorbed becomes smaller and the light transmittance usually becomes larger. The increase in the light transmittance increases light absorbance in the photoelectric conversion film and increases the photoelectric conversion ability, which is preferable. Considering suppression of leakage current, an increase in a resistance value of a thin film, and an increase in transmittance accompanied by the thinning, the film thickness of the upper electrode 15 is preferably 5 to 100 nm and more preferably 5 to 20 nm.

There is a case where the lower electrode 11 has a transparency depending on the application or an opposite case where the lower electrode does not have transparency and reflects light. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO), metals such as gold, silver, chromium, nickel, titanium, tungsten, and aluminum, conductive compounds (for example, titanium nitride (TiN)) such as oxides or nitrides of these metals, mixtures or laminates of these metals and conductive metal oxides, and organic conductive materials such as polyaniline, polythiophene, and polypyrrole.

The method for forming electrodes is not particularly limited, and can be appropriately selected in accordance with the electrode material. Specific examples thereof include a printing method, a wet method such as a coating method, physical methods such as a vacuum vapor deposition method, a sputtering method, and an ion plating method, and chemical methods such as a CVD method and a plasma CVD method.

In a case where the material of the electrodes is ITO, examples thereof include an electron beam method, a sputtering method, a resistance thermal vapor deposition method, a chemical reaction method (such as a sol-gel method), and a coating method with a dispersion of indium tin oxide.

[Charge Blocking Film: Electron Blocking Film, Hole Blocking Film]

The photoelectric conversion element of the present invention may have a charge blocking film. In the case where the photoelectric conversion element of the present invention has this film, the characteristics (such as photoelectric conversion efficiency and response speed) of photoelectric conversion element to be obtained become superior. Examples of the charge blocking film include an electron blocking film and a hole blocking film. Hereinafter, the films will be described in detail.

[Electron Blocking Film]

The electron blocking film includes an electron donating compound. Specific examples of a low molecular material include aromatic diamine compounds such as N,N′-bis(3-methylphenyl)-(1,1′-biphenyl)-4,4′-diamine (TPD) and 4,4′-bis[N-(naphthyl)-N-phenyl-amino]biphenyl (α-NPD), porphyrin compounds such as porphyrin, copper tetraphenylporphyrin, phthalocyanine, copper phthalocyanine, and titanium phthalocyanine oxide, oxazole, oxadiazole, triazole, imidazole, imidazolone, a stilbene derivative, a pyrazoline derivative, tetrahydroimidazole, polyarylalkane, butadiene, 4,4′,4″-tris(N-(3-methylphenyl)-N-phenylamino) triphenylamine (m-MTDATA), a triazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, and a silazane derivative, and specific examples of a polymer material include a polymer such as phenylenevinylene, fluorene, carbazole, indole, pyrene, pyrrole, picoline, thiophene, acetylene, and diacetylene, or a derivative thereof.

The electron blocking film may be formed of a plurality of films.

The electron blocking film may be formed of an inorganic material. In general, an inorganic material has a dielectric constant larger than that of an organic material. Therefore, in a case where the inorganic material is used in the electron blocking film, a large voltage is applied to the photoelectric conversion film, and therefore, the photoelectric conversion efficiency increases. Examples of the inorganic material that can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

(Hole Blocking Film)

The hole blocking film includes an electron accepting compound.

Examples of the electron accepting compound include an oxadiazole derivative such as 1,3-bis(4-tert-butylphenyl-1,3,4-oxadiazolyl)phenylene (OXD-7), an anthraquinodimethane derivative, a diphenylquinone derivative, bathocuproine, bathophenanthroline, and derivatives thereof, a triazole compound, a tris(8-hydroxyquinolinato)aluminum complex, a bis(4-methyl-8-quinolinato)aluminum complex, a distyrylarylene derivative, and a silole compound.

A method for manufacturing the charge blocking film is not particularly limited, and examples thereof include a dry film formation method or a wet film formation method. Examples of the dry film formation method include a vapor deposition method and a sputtering method. Any one of the physical vapor deposition (PVD) or chemical vapor deposition (CVD) may be used for the vapor deposition, but physical vapor deposition such as vacuum vapor deposition is preferable. Examples of the wet film formation method include an inkjet method, a spray method, a nozzle printing method, a spin coating method, a dip coating method, a casting method, a die coating method, a roll coating method, a bar coating method, and a gravure coating method, and an inkjet method is preferable from the viewpoint of high precision patterning.

The thickness of the charge blocking film (the electron blocking film and the hole blocking film) is preferably 10 to 200 nm, more preferably 30 to 150 nm, and still more preferably 50 to 100 nm.

[Substrate]

The photoelectric conversion element may further include a substrate. The type of substrate to be used is not particularly limited, and examples thereof include a semiconductor substrate, a glass substrate, and a plastic substrate.

The position of the substrate is not particularly limited, but in general, a conductive film, a photoelectric conversion film, and a transparent conductive film are laminated on the substrate in this order.

[Sealing Layer]

The photoelectric conversion element may further include a sealing layer. The performance of the photoelectric conversion material may deteriorate noticeably due to the presence of deterioration factors such as water molecules. The deterioration can be prevented by sealing and coating the entirety of the photoelectric conversion film with a sealing layer such as diamond-like carbon (DLC) or ceramics such as metal oxide, metal nitride, and metal nitride oxide which are dense and into which water molecules do not permeate.

Selection of the material of the sealing layer and manufacture of the sealing layer may be performed in accordance with the disclosure in paragraphs <0210> to <0215> in JP2011-082508A.

[Optical Sensor]

Examples of the application of the photoelectric conversion element include a photoelectric cell and an optical sensor, but the photoelectric conversion element of the present invention is preferably used as an optical sensor. The above-described photoelectric conversion element alone may be used in an optical sensor. Alternately, the above-described photoelectric conversion element may be used in a line sensor in which photoelectric conversion elements described above are linearly arranged or a two-dimensional sensor in which the photoelectric conversion elements are arranged on a plane. In the line sensor, the photoelectric conversion element of the present invention functions as an imaging element by converting optical image information into an electric signal using a driving unit and an optical system such as a scanner. In the two-dimensional sensor, the photoelectric conversion element of the present invention functions as an imaging element by converting optical image information into an electric signal by imaging the optical image information on the sensor in an optical system such as an imaging module.

[Imaging Element]

Next, a configuration example of an imaging element including the photoelectric conversion element 10a will be described.

In the configuration example which will be described below, the same reference numerals or the corresponding reference numerals are attached to members or the like having the same configuration or action as those which have already been described, to simplify or not to repeat the description.

The imaging element is an element that converts optical information of an image into an electric signal, and is an element in which a plurality of photoelectric conversion elements are arranged on a matrix in the same plane, optical signals are converted into electric signals in each photoelectric conversion element (pixel), and it is possible to sequentially output the electric signals to the outside of the imaging elements for each pixel. For this reason, one pixel is formed of one photoelectric conversion element and one or more transistors.

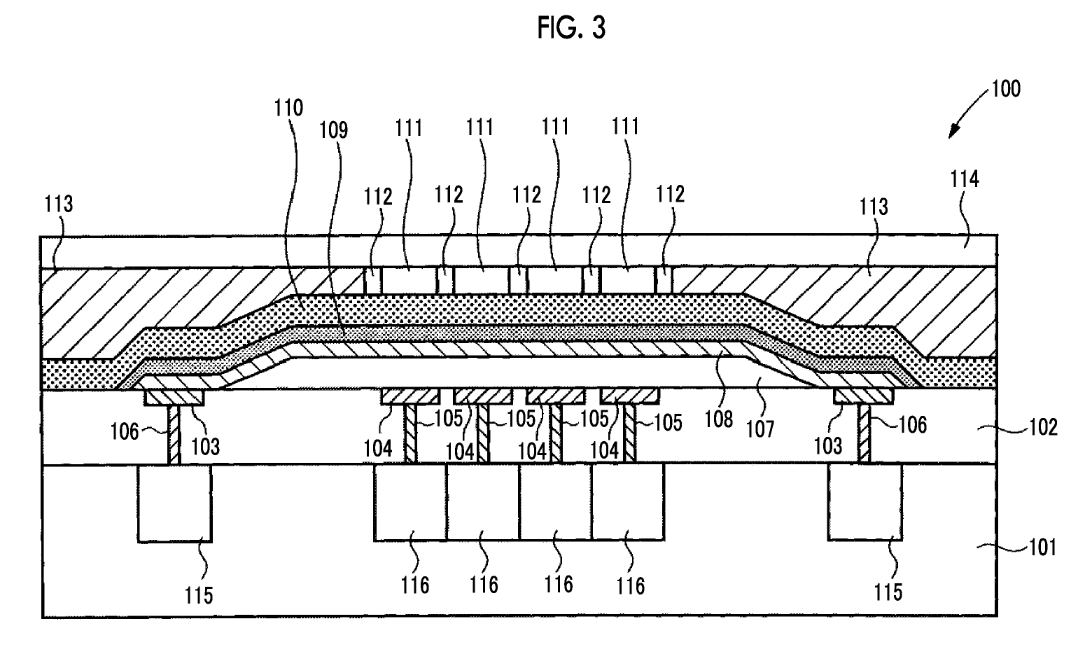

FIG. 3 is a schematic cross-sectional view showing a schematic configuration of an imaging element for describing an embodiment of the present invention. This imaging element is mounted on an imaging device such as a digital camera and a digital video camera, and imaging modules such as an electronic endoscope and a cellular phone.

The imaging element has a plurality of photoelectric conversion elements having configurations shown in FIG. 1A and a circuit substrate in which a readout circuit reading out a signal corresponding to charges generated in a photoelectric conversion film of each photoelectric conversion element is formed. The imaging element has a configuration in which the plurality of photoelectric conversion elements are one-dimensionally or two-dimensionally arranged on the same surface above the circuit substrate.

An imaging element 100 shown in FIG. 3 includes a substrate 101, an insulating layer 102, connection electrodes 103, pixel electrodes (lower electrodes) 104, connection units 105, connection units 106, a photoelectric conversion film 107, a counter electrode (upper electrode) 108, a buffer layer 109, a sealing layer 110, a color filter (CF) 111, partition walls 112, a light shielding layer 113, a protective layer 114, a counter electrode voltage supply unit 115, and readout circuits 116.

The pixel electrodes 104 have the same function as that of the lower electrode 11 of the photoelectric conversion element 10a shown in FIG. 1A. The counter electrode 108 has the same function as that of the upper electrode 15 of the photoelectric conversion element 10a shown in FIG. 1A. The photoelectric conversion film 107 has the same configuration as that of the layer provided between the lower electrode 11 and the upper electrode 15 of the photoelectric conversion element 10a shown in FIG. 1A.

The substrate 101 is a glass substrate or a semiconductor substrate of Si or the like. The insulating layer 102 is formed on the substrate 101. A plurality of pixel electrodes 104 and a plurality of connection electrodes 103 are formed on the surface of the insulating layer 102.

The photoelectric conversion film 107 is a layer common to all the photoelectric conversion elements provided so as to cover the plurality of pixel electrodes 104.

The counter electrode 108 is an electrode common to all the photoelectric conversion elements provided on the photoelectric conversion film 107. The counter electrode 108 is formed on the connection electrodes 103 arranged on an outer side than the photoelectric conversion film 107, and is electrically connected to the connection electrodes 103.

The connection units 106 are buried in the insulating layer 102, and are plugs for electrically connecting the connection electrodes 103 to the counter electrode voltage supply unit 115. The counter electrode voltage supply unit 115 is formed in the substrate 101 and applies a predetermined voltage to the counter electrode 108 via the connection units 106 and the connection electrodes 103. In a case where a voltage to be applied to the counter electrode 108 is higher than a power supply voltage of the imaging element, the power supply voltage is boosted by a boosting circuit such as a charge pump to supply the predetermined voltage.

The readout circuits 116 are provided on the substrate 101 corresponding to each of the plurality of pixel electrodes 104, and reads out a signal corresponding to charges trapped by the corresponding pixel electrodes 104. The readout circuits 116 are constituted, for example, of CCD and CMOS circuits or a thin film transistor (TFT) circuit, and are shielded by a light shielding layer not shown in the drawing which is disposed in the insulating layer 102. The readout circuits 116 are electrically connected to the corresponding the pixel electrodes 104 via the connection units 105.

The buffer layer 109 is formed on the counter electrode 108 so as to cover the counter electrode 108. The sealing layer 110 is formed on the buffer layer 109 so as to cover the buffer layer 109. Color filters 111 are formed on the sealing layer 110 at positions facing each of the pixel electrodes 104. The partition walls 112 are provided between the color filters 111 and are used for improving the light transmission efficiency of the color filters 111.

The light shielding layer 113 is formed on the sealing layer 110 in a region other than the region where the color filters 111 and the partition walls 112 are provided, and prevent light from entering the photoelectric conversion film 107 formed outside an effective pixel region. The protective layer 114 is formed on the color filters 111, the partition walls 112, and the light shielding layer 113, and protects the entirety of the imaging element 100.

In the imaging element 100 configured as described above, light which has entered is incident on the photoelectric conversion film 107, and charges are generated here. Holes among the generated charges are trapped by the pixel electrodes 104, and voltage signals corresponding to the amount are output to the outside of the imaging element 100 using the readout circuits 116.

A method for manufacturing the imaging element 100 is as follows.

The connection units 105 and 106, the plurality of connection electrodes 103, the plurality of pixel electrodes 104, and the insulating layer 102 are formed on the circuit substrate in which the counter electrode voltage supply unit 115 and the readout circuits 116 are formed. The plurality of pixel electrodes 104 are disposed, for example, in a square lattice shape on the surface of the insulating layer 102.

Next, the photoelectric conversion film 107 is formed on the plurality of pixel electrodes 104, for example, through a vacuum vapor deposition method. Next, the counter electrode 108 is formed on the photoelectric conversion film 107 under vacuum, for example, through a sputtering method. Next, the buffer layer 109 and the sealing layer 110 are sequentially formed on the counter electrode 108, through the vacuum vapor deposition method. Next, after forming the color filters 111, the partition walls 112, and the light shielding layer 113, the protective layer 114 is formed, and the imaging element 100 is completed.

### Examples

Examples will be shown below, but the present invention is not limited thereto.

(Synthesis of Compound (D-3))

A compound (D-3) was synthesized according to the following scheme.

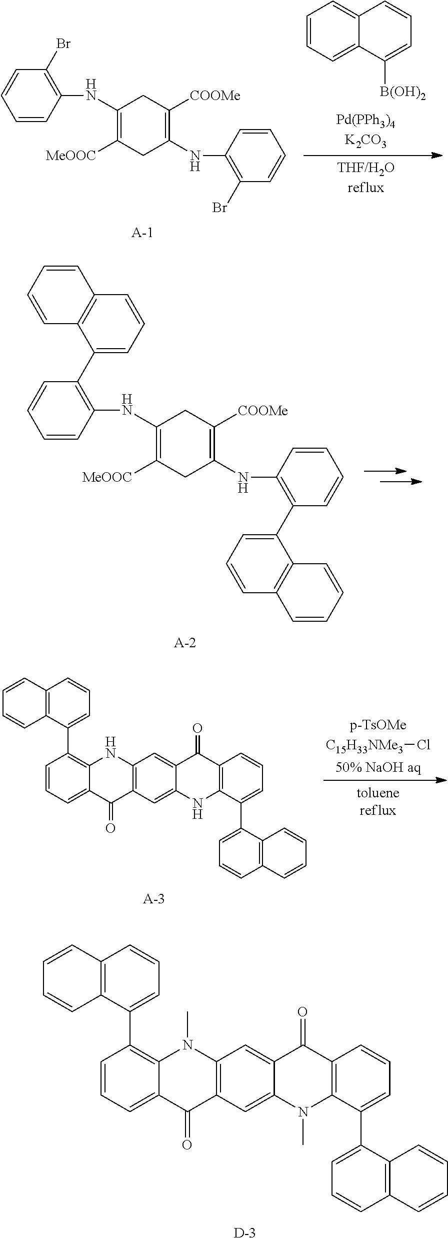

A compound (A-1) was synthesized through the same method as that with the conditions disclosed in JP2011-026317A.

The compound (A-1) (7.00 g, 13.1 mmol), 1-naphthylboronic acid (6.74 g, 39.1 mmol), and potassium carbonate (9.05 g, 65.5 mmol) were added to a mixed solution of tetrahydrofuran (200 mL) and water (10 mL), and evacuation and nitrogen substitution were repeated to perform deaeration. Tetrakis (triphenylphosphine) palladium (0) (1.51 g, 1.31 mmol) was added to the obtained solution for performing a reaction for 6 hours by heating and refluxing the solution. After allowing the solution to cool, an aqueous ammonium chloride solution and ethyl acetate were added to the solution, and liquid separation treatment was performed to separate an organic phase from the solution. After adding magnesium sulfate to the separated organic phase, filtration treatment was performed, and the obtained filtrate was concentrated. Thereafter, the obtained crude product was recrystallized from ethanol to obtain a compound (A-2) (6.44 g, yield of 78%).

A compound (A-3) was synthesized through the same method as that disclosed in JP2011-026317A using the obtained compound.

The compound (A-3) (1.13 g, 2.00 mmol), trimethylhexadecylammonium chloride (640 mg, 2.00 mmol), and methyl p-toluenesulfonate (1.86 g, 10.0 mmol) were added to toluene (100 mL). A 50 mass % aqueous sodium hydroxide solution (5.0 mL) was added thereto while stirring the obtained solution at room temperature. The obtained solution was heated and refluxed and was reacted for 6 hours. Then, the reactant was allowed to cool. The precipitated solid was collected through filtration, and the collected solid was washed with water and methanol. The obtained solid was dispersed in and washed with methanol for 3 hours, and was then collected through filtration. The obtained solid was dispersed in and washed with tetrahydrofuran to obtain a compound (D-3) (0.65 g, yield of 55%). The obtained compound (D-3) was identified through nuclear magnetic resonance (NMR) and mass spectrometry (MS).

¹H NMR (400 MHz, CDCl₃): δ=3.18 (s, 6H), 7.39 (t, 4H), 7.45-7.65 (m, 8H), 7.75 (d, 2H), 7.98 (t, 4H), 8.35 (s, 2H), 8.62 (d, 2H)

MS (ESI⁺) m/z: 593.2 ([M+H]⁺)

Hereinafter, compounds (D-1), (D-2), and (D-4) to (D-14) and compounds (R-2) and (R-3) were also synthesized using the same reaction.

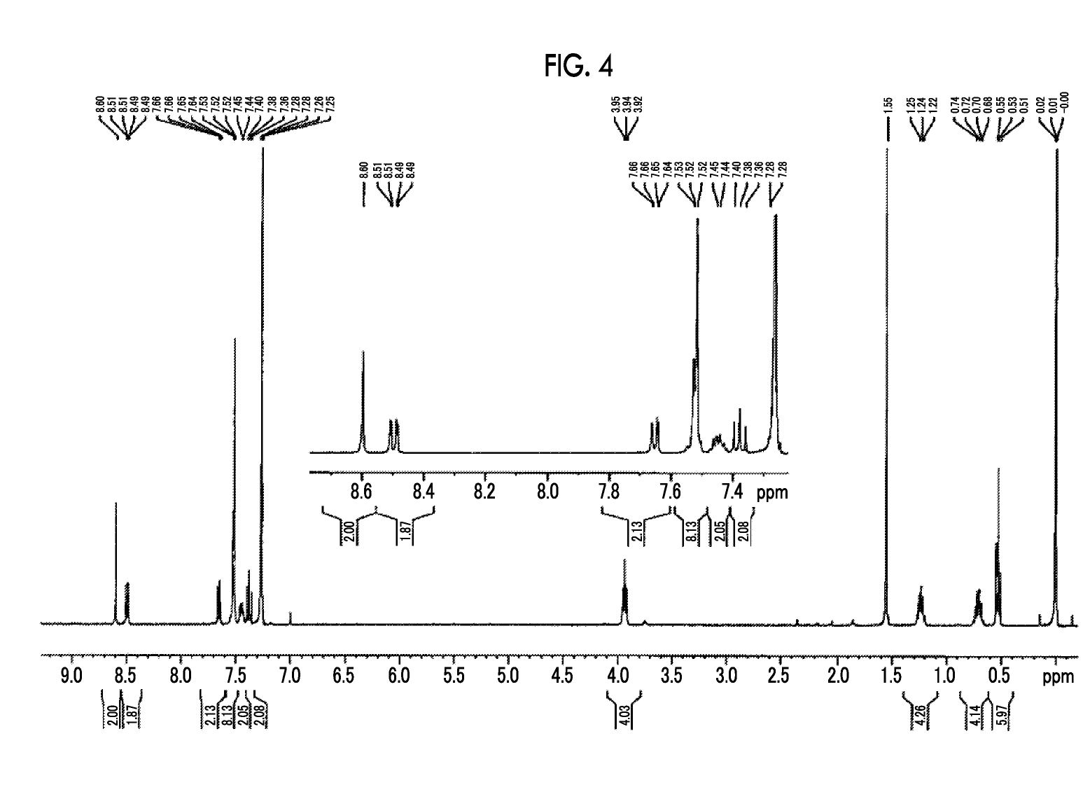

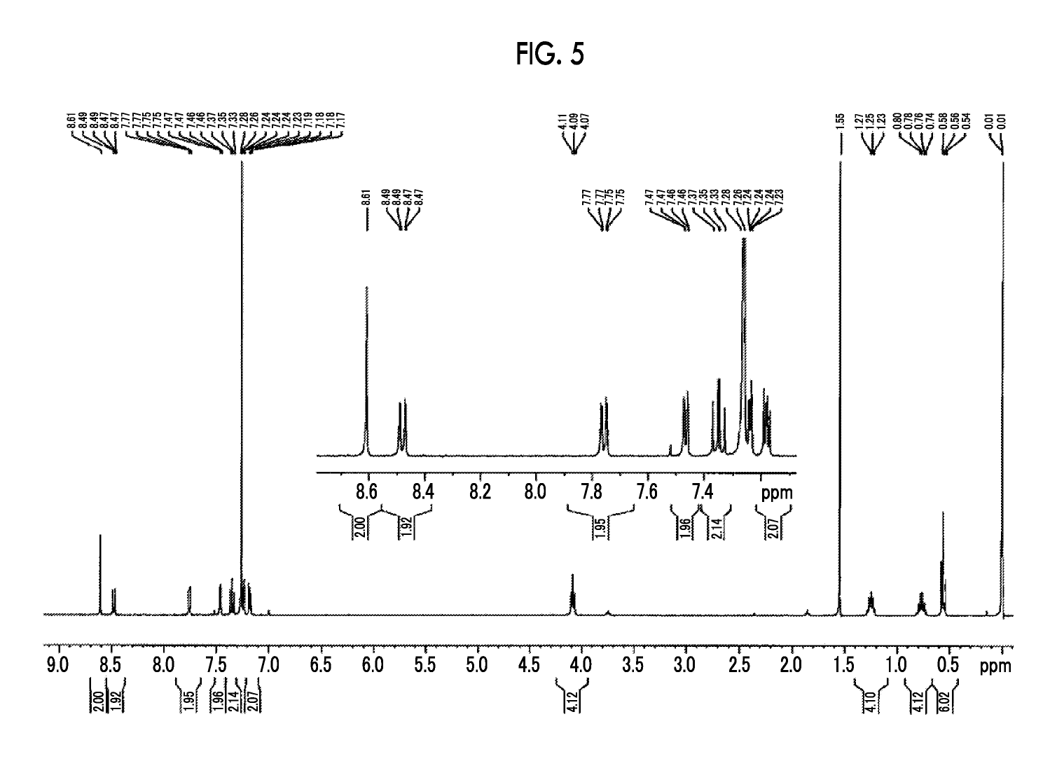

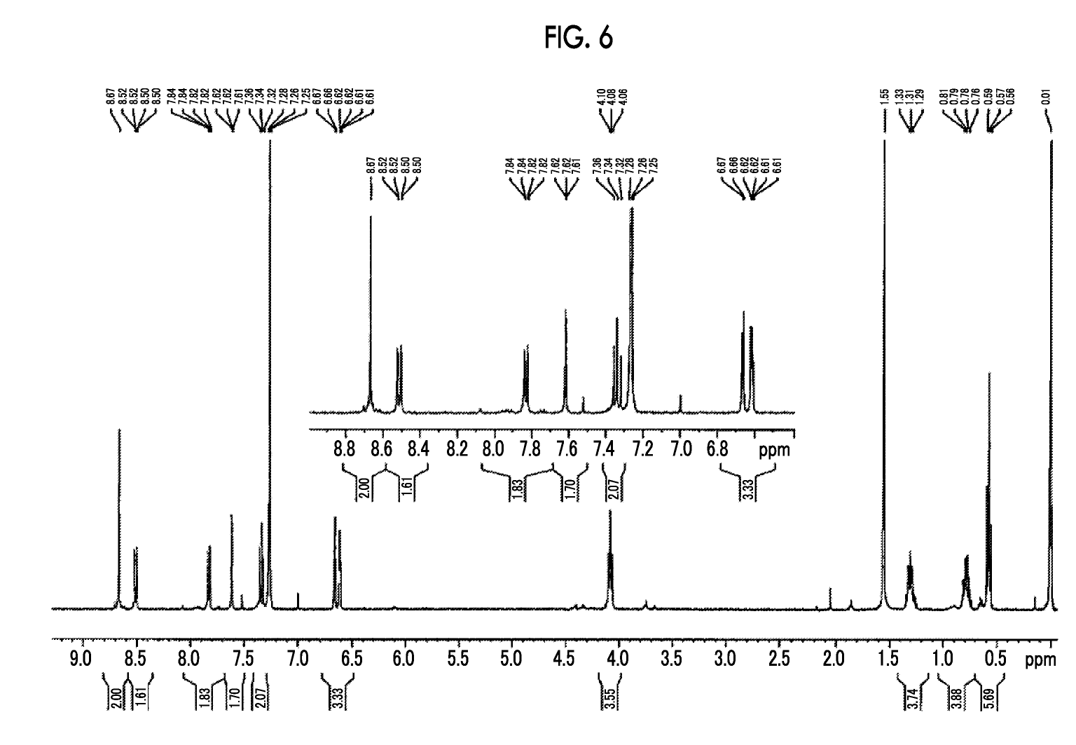

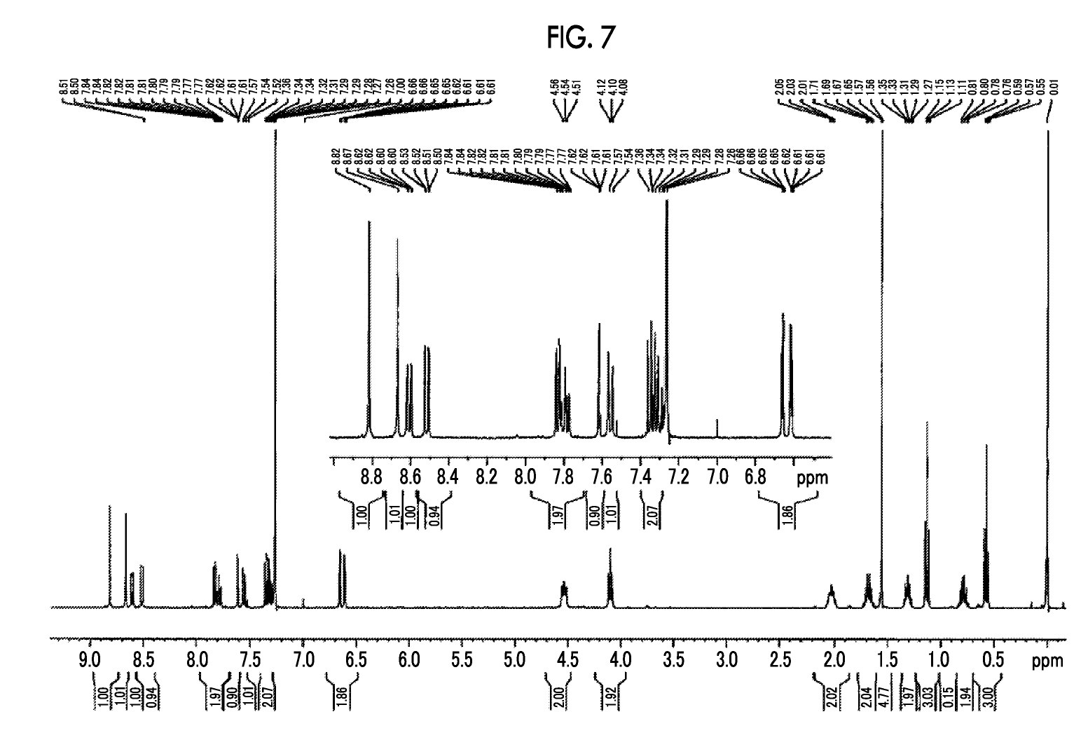

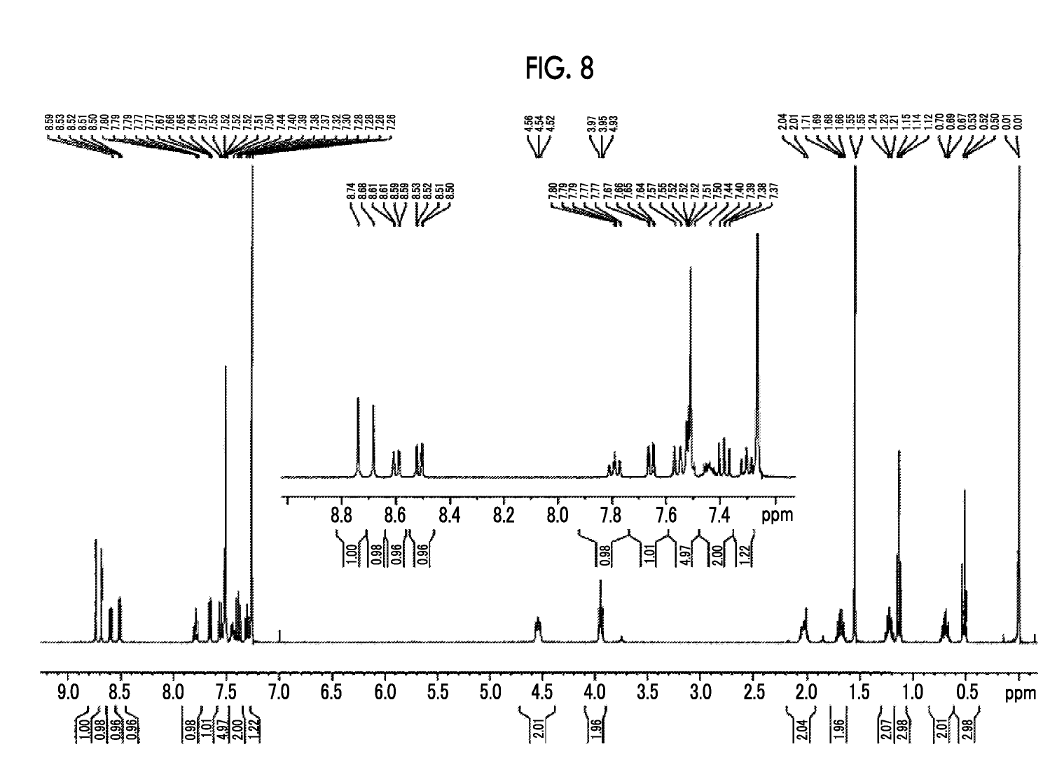

¹H NMR (solvent: CDCl₃) spectra of the compounds (D-10) to (D-14) are shown in FIGS. 4 to 8, respectively.

A compound (R-1) corresponding to a comparative compound was purchased from Luminescence Technology.

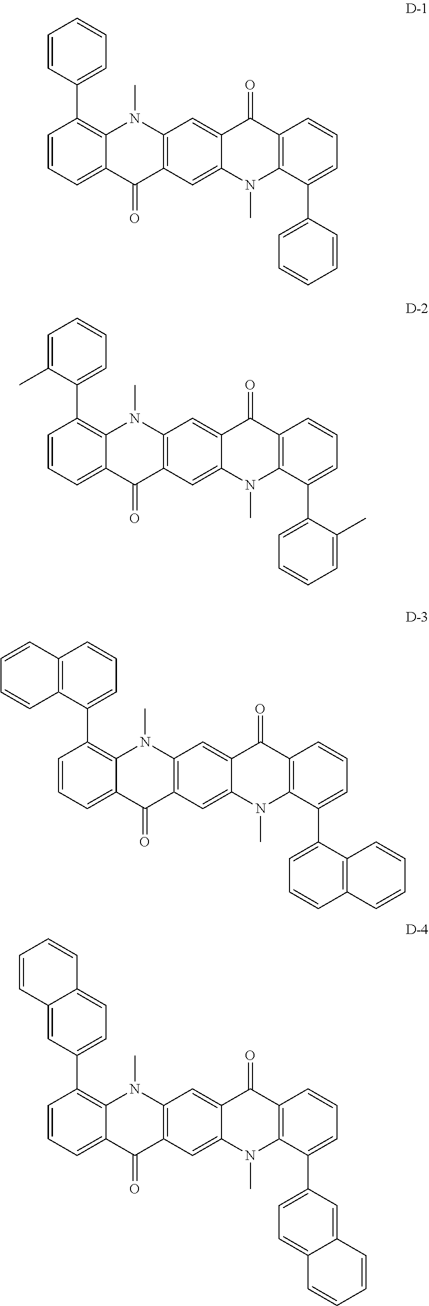

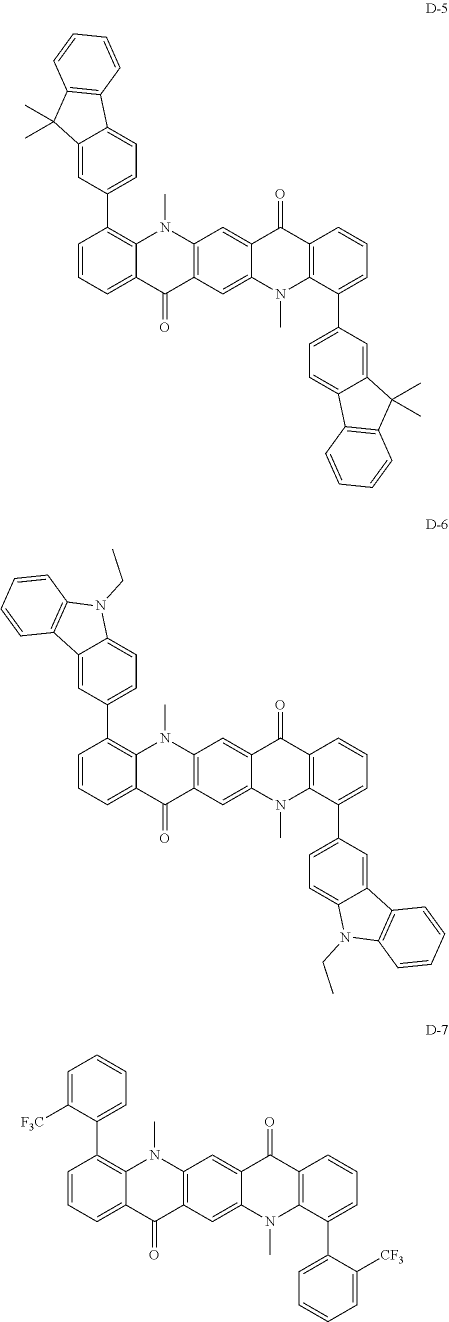

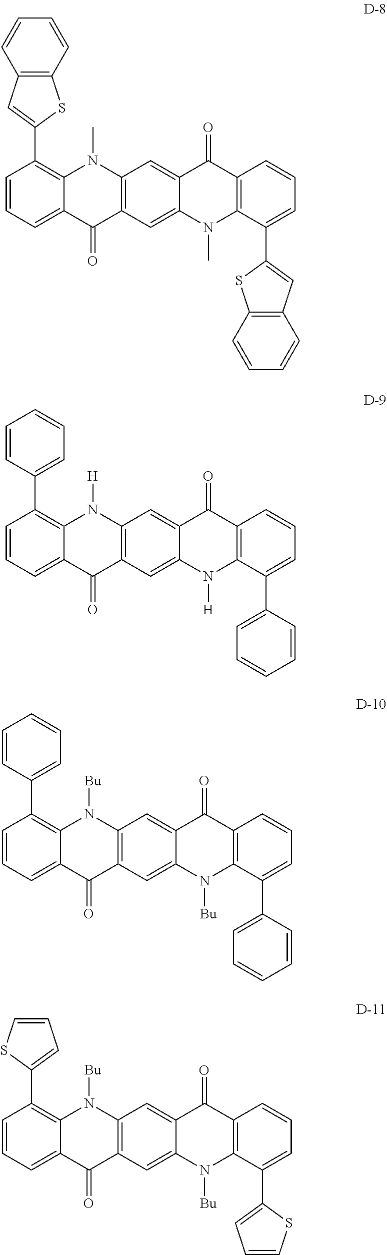

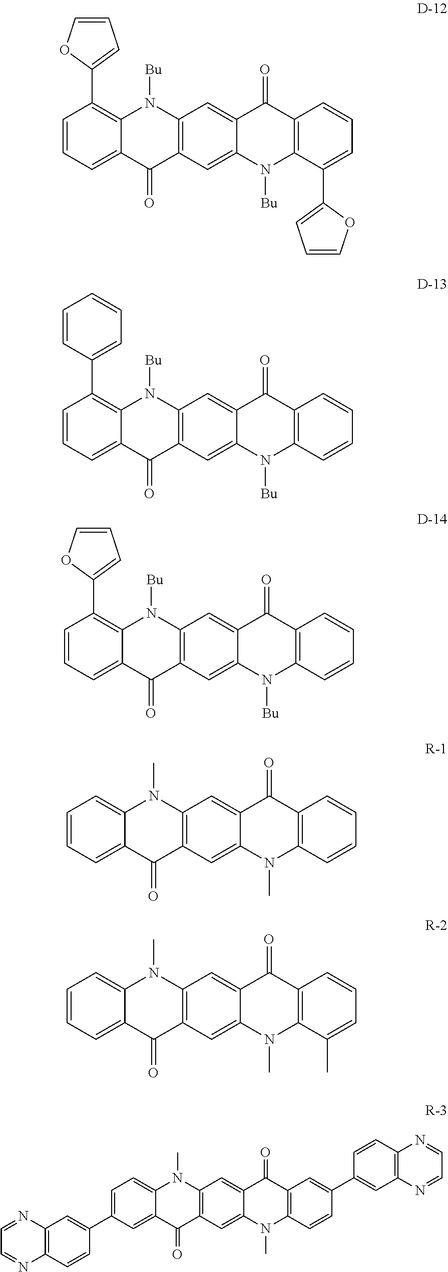

<Production of Photoelectric Conversion Element>

A photoelectric conversion element in a form of FIG. 1A was produced using the obtained compounds. Hereinafter, a case where the compound (D-1) is used will be described in detail.

Specifically, an amorphous ITO film was formed on a glass substrate through a sputtering method to form a lower electrode 11 (thickness: 30 nm). Furthermore, a film of molybdenum oxide (MoO_{X}) was formed on the lower electrode 11 through a vacuum vapor deposition method to form a molybdenum oxide layer (thickness: 60 nm) as an electron blocking film 16A.

Furthermore, the compound (D-1) and the following compound (N-1) were subjected to co-vapor deposition so as to be respectively 50 nm in terms of a single layer so as to form a film on a molybdenum oxide layer in a state where the temperature of the substrate was controlled to be 25° C., and the photoelectric conversion film 12 having a bulk hetero structure of 100 nm was formed.

Furthermore, an amorphous ITO film was formed on the photoelectric conversion film 12 through a sputtering method to form the upper electrode 15 (transparent conductive film) (thickness: 10 nm). After forming a SiO film on the upper electrode 15 as a sealing layer through thermal vapor deposition, an aluminum oxide (Al₂O₃) layer was formed thereon through atomic layer chemical vapor deposition (ALCVD) method to produce a photoelectric conversion element.

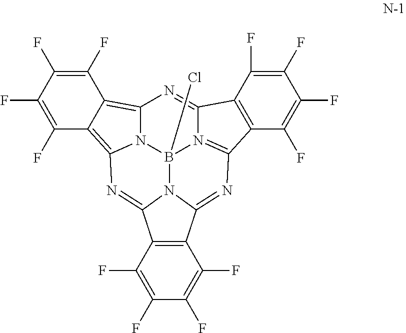

A photoelectric conversion element of each example was produced according to the same procedure as above except that the above-described compound (D-1) was changed to compounds (D-2) to (D-14) and compounds (R-1) to (R-3).

<Evaluation>

(Evaluation of Responsiveness)

The following evaluation of responsiveness was performed using each obtained photoelectric conversion element.

Specifically, a voltage was applied to each photoelectric conversion element so that the intensity became 1.0×10⁵ V/cm. Thereafter, light emitting diode (LED) was instantaneously turned on to radiate light from the upper electrode (transparent conductive film) side. The photocurrent at that time was measured with an oscilloscope to obtain a rise time between signal intensities of 0% to 97%. Then, the relative value when the rise time in Comparative Example 1 was set to 10 was obtained. The results are shown in Table 1.

A case where the relative value of the rise time is less than 3 with respect to Comparative Example 1 is set as “A”, a case where the relative value thereof is greater than or equal to 3 and less than 5 is set as “B”, a case where the relative value thereof is greater than or equal to 5 and less than 10 is set as “C”, and a case where the relative value thereof is greater than or equal to 10 is set as “D”. For practical use, “A” or “B” is preferable, and “A” is more preferable.

TABLE 1

| | Compound | | | |
|---|---|---|---|---|
| | | Molecular | Evaluation | |
| Example | Type | weight | Responsiveness | Remarks |
| 1 | D-1 | 493 | A | Example |
| 2 | D-2 | 521 | A | Example |
| 3 | D-3 | 593 | A | Example |
| 4 | D-4 | 593 | A | Example |
| 5 | D-5 | 725 | A | Example |
| 6 | D-6 | 727 | A | Example |
| 7 | D-7 | 629 | A | Example |
| 8 | D-8 | 605 | A | Example |
| 9 | D-9 | 465 | B | Example |
| 10 | D-10 | 577 | A | Example |
| 11 | D-11 | 589 | A | Example |
| 12 | D-12 | 557 | A | Example |
| 13 | D-13 | 501 | B | Example |
| 14 | D-14 | 491 | B | Example |
| 15 | R-1 | 340 | D | Comparative |
| | | | | Example |
| 16 | R-2 | 368 | C | Comparative |
| | | | | Example |
| 17 | R-3 | 569 | C | Comparative |
| | | | | Example |

As shown in Table 1, it was confirmed that the photoelectric conversion elements of the present invention exhibit excellent performance (responsiveness and heat resistance).

Among these, it was confirmed from a comparison between Example 9 and Examples 1 to 8 that, in a case where B¹ and B² are alkyl groups, the effect is superior.

A desired effect was not obtained in Comparative Examples 1 to 3 in which a predetermined compound was not used. The compound used in Comparative Example 3 corresponds to the compound specifically disclosed in US2014/0097416A.

<Production of Imaging Element>

The same imaging element as that shown in FIG. 3 was produced. That is, a 30 nm amorphous TiN film was formed on a CMOS substrate through a sputtering method, and was then used as a lower electrode through patterning such that each pixel was present on a photodiode (PD) on the CMOS substrate through photolithography. The imaging element was produced similarly to Examples 1 to 14 after the film formation of the electron blocking material. Evaluation of responsiveness of each of the obtained imaging elements was also carried out in the same manner, and the same results as those in Table 1 were obtained. It was found that each of the imaging elements also exhibited excellent performance.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: hole blocking film
100: imaging element
101: substrate
102: insulating layer
103: connection electrode
104: pixel electrode (lower electrode)
105: connection unit
106: connection unit
107: photoelectric conversion film
108: counter electrode (upper electrode)
109: buffer layer
110: sealing layer
111: color filter (CF)
112: partition wall
113: light shielding layer
114: protective layer
115: counter electrode voltage supply unit
116: readout circuit
200: photoelectric conversion element (hybrid type photoelectric conversion element)
201: inorganic photoelectric conversion film
202: n-type well
203: p-type well
204: n-type well
205 p-type silicon substrate
207: insulating layer
208: pixel electrode
209: organic photoelectric conversion film
210: common electrode
211: protective film
212: electron blocking film

What is claimed is:

1. A photoelectric conversion element having a conductive film, a photoelectric conversion film, and a transparent conductive film in this order,
wherein the photoelectric conversion film contains a compound represented by Formula (1), 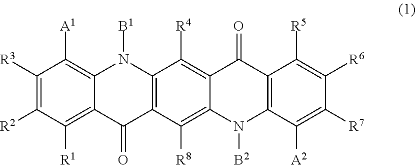
in Formula (1), R¹ to R⁸ each independently represent a hydrogen atom or a substituent, B¹ and B² each independently represent an alkyl group or a heteroaryl group, A¹ and A² each independently represent a hydrogen atom or a substituent, at least one of A¹ or A² represents an aryl group or a heteroaryl group, and adjacent groups among R¹ to R⁸, A¹, A², B¹, and B² may be linked to each other to form a ring.

2. The photoelectric conversion element according to claim 1,
wherein, in Formula (1), both A¹ and A² represent an aryl group or a heteroaryl group.

3. The photoelectric conversion element according to claim 1,
wherein, in Formula (1), B¹ and B² represent an alkyl group.

4. The photoelectric conversion element according to claim 2,
wherein, in Formula (1), B¹ and B² represent an alkyl group.

5. The photoelectric conversion element according to claim 1,
wherein a molecular weight of the compound represented by Formula (1) is 470 to 900.

6. The photoelectric conversion element according to claim 1,
wherein the photoelectric conversion film further includes an n-type organic semiconductor.

7. The photoelectric conversion element according to claim 1,
wherein the photoelectric conversion film further includes a p-type organic semiconductor.

8. The photoelectric conversion element according to claim 1, further comprising:
an electron blocking film.

9. The photoelectric conversion element according to claim 1, further comprising:
a hole blocking film.

10. An optical sensor comprising:
the photoelectric conversion element according to claim 1.

11. An imaging element comprising:
the photoelectric conversion element according to claim 1.

12. A compound represented by Formula (2), 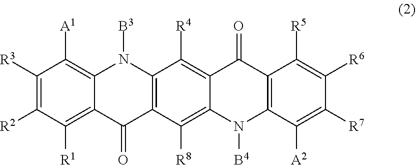
in Formula (2), R¹ to R⁸ each independently represent a hydrogen atom or a substituent, B³ and B⁴ each independently represent an alkyl group or a heteroaryl group, A¹ and A² each independently represent a hydrogen atom or a substituent, at least one of A¹ or A² represents an aryl group or a heteroaryl group, and adjacent groups among R¹ to R⁸, A¹, A², B³, and B⁴ may be linked to each other to form a ring.

13. The compound according to claim 12,
wherein, in Formula (2), both A¹ and A² represent an aryl group or a heteroaryl group.

14. The compound according to claim 12,
wherein, in Formula (2), B³ and B⁴ represent an alkyl group.

15. The compound according to claim 13,
wherein, in Formula (2), B³ and B⁴ represent an alkyl group.
